(19) United States
(12) Patent Application Publication
(10) Pub. No.: US 2023/0142825 A1
(43) Pub. Date: May 11, 2023

(54) THERAPEUTIC METHOD FOR THE EYE USING ULTRASOUND

(71) Applicant: ArcScan, Inc., Golden, CO (US)

(72) Inventors: Johan E. Giphart, Superior, CO (US); Andrew K. Levien, Morrison, CO (US); John David Watson, Evergreen, CO (US)

(21) Appl. No.: 17/983,200

(22) Filed: Nov. 8, 2022

Related U.S. Application Data
(60) Provisional application No. 63/277,012, filed on Nov. 8, 2021.

(51) Int. Cl. A61B 8/10; A61F 9/007

(52) U.S. Cl. CPC A61B 8/10; A61F 9/00745

(57) ABSTRACT

The present disclosure relates to ultrasound imaging and treatment of an eye and in particular directed to an apparatus and method for reducing intraocular pressure by 1) ablating the ciliary process which is the structure responsible for production of aqueous humor and 2) by vibrating the trabecular mesh to stimulate better drainage of fluid through the trabecular mesh and out of the eye. The present disclosure describes an apparatus and method for forming a high precision image of the eye wherein the resolution is sufficient to image, for example, ciliary body and region around the trabecular mesh. The present disclosure further discloses an imaging transducer and an irradiating therapeutic transducer that can be mounted such that they are movable between a plurality of positions.

### CROSS-REFERENCE TO RELATED APPLICATIONS

This application claims the benefits, under 35 U.S.C.§ 119(e), of Provisional Application Ser. No. 63/277,012 entitled “Ultrasound Scanning and Therapeutic Apparatus and Method” filed Nov. 8, 2021, which is incorporated herein by reference.

### FIELD

The present disclosure relates to ultrasound imaging and treatment of a human eye and in particular directed to an apparatus and method for reducing intraocular pressure by 1) ablating the ciliary process which is the structure responsible for production of aqueous humor and by 2) vibrating or undulating the trabecular mesh to stimulate better drainage of fluid through the trabecular mesh and out of the eye.

### BACKGROUND

Ultrasonic imaging has found use in accurate and reproducible measurements of structures of the eye, such as, for example, the cornea and lens capsule. Such measurements provide an ophthalmic surgeon valuable information that can be used to guide various surgical procedures for correcting refractive errors such as LASIK and lens replacement. They also provide diagnostic information after surgery has been performed to assess the geometrical location of corneal features such as the LASIK scar and lens features such as lens connection, position and orientation. This allows the surgeon to assess post surgical changes in the cornea or lens and to take steps to correct any problems that develop.

Except for on-axis measurements, dimensions and locations of eye components behind the iris cannot be fully determined by optical means. Ultrasonic imaging in the frequency range of about 5 MHz to about 80 MHz can be applied to make accurate and precise measurements of structures of the eye, such as the cornea, lens capsule, ciliary muscle and the like.

An ultrasound scanning apparatus is described in the following issued US patents, all of which are incorporated herein by reference:

1. U.S. Pat. No. 7,048,690 “Precision Ultrasound Measurement for Intraocular Lens Placement”;
2. U.S. Pat. No. 8,758,252 “Innovative Components for an Ultrasonic Arc Scanning Apparatus”;
3. U.S. Pat. No. 8,496,588 “Procedures for an Ultrasonic Arc Scanning Apparatus”
4. U.S. Pat. No. 8,317,709 “Alignment and Imaging of an Eye with an Ultrasonic Scanner”
5. U.S. Pat. No. 9,149,254 “Alignment and Imaging of an Eye with an Ultrasonic Scanner”
6. U.S. Pat. No. 9,597,059 “Tracking Unintended Eye Movements in an Ultrasonic Scan of the Eye”
7. U.S. Pat. No. 9,320,427 “Combination Optical and Ultrasonic Imaging of an Eye”
8. U.S. Pat. No. 10,736,605 Disposable Eyepiece System for an Ultrasonic Eye Scanning Apparatus

An arc scanner is an ultrasound scanning device utilizing a transducer that both sends and receives pulses as it moves along an arcuate guide track. The arcuate guide track has a center of curvature whose position can be moved to scan different curved surfaces. Later versions of arc scanners have mechanisms that allow the radius of curvature of the scanner to be changed. In this type of scanner, a transducer is moved along an arcuate guide track whose center of curvature can be changed and set approximately at the center of curvature of the eye surface of interest. The transducer generates many acoustic pulses as it moves along the arcuate guide track. These pulses reflect off specular surfaces and other tissue interfaces within the eye. Each individual return pulse is detected and digitized to produce a series of A-scans. The A-scans can then be combined to form a cross-sectional image of the eye features of interest. The image combining A-scans is commonly called a B scan.

At a center frequency of about 38 MHz, a typical arc scanner has an axial resolution of about 20 microns and a lateral resolution of about 150 microns. The reproducibility of arc scanner images is typically about 2 microns.

The ultrasonic system described herein is capable of accurately moving an ultrasound transducer with respect to a known reference point on a patient's head. Further improvements allow for tracking of unintended eye motions during scanning as disclosed in U.S. Pat. No. 9,597,059 entitled “Tracking Unintended Eye Movements in an Ultrasonic Scan of the Eye”.

Ultrasonic imaging requires a liquid medium to be interposed between the object being imaged and the transducer, which requires, in turn, that the eye, the transducer and the path between them be at all times immersed in a liquid medium. Concern for safety of the cornea introduces the practical requirement that the liquid medium be either pure water or normal saline water solution. There are reasons to prefer that the medium be pure water or physiologic saline (also known as normal saline) but the embodiments do not exclude other media suitable for conducting acoustic energy in the form of ultrasound. Most other media present an increased danger to the patient's eye, even with a barrier interposed between the eye and the ultrasonic transducer. Barriers can leak or be breached, allowing the liquids on either side to mix, thus bringing a potentially harmful material into contact with the eye.

An eyepiece serves to complete a continuous acoustic path for ultrasonic scanning, that path extending from the transducer to the surface of the patient's eye. The eyepiece also separates the water in which the patient's eye is immersed from the water in the chamber in which the ultrasound transducer and guide track assembly are contained. Finally, the eyepiece provides a steady rest for the patient and helps the patient to remain steady during a scan. To be practical, the eyepiece should be free from frequent leakage problems, should be comfortable to the patient and its manufacturing cost should be low since it should be replaced for every new patient.

Another ultrasound scanning method is known as Ultrasound Bio Microscopy as embodied in a hand-held device commonly known as a UBM. A UBM can capture anterior segment images using a transducer to emit short acoustic pulses ranging from about 20 to about 80 MHz. This type of ultrasound scanner is also called a sector scanner.

A UBM is a hand-held ultrasonic scanner whose beams sweep a sector like a radar transmitter. In this type of scanner, an ultrasonic transducer oscillates about a fixed position so as to produce many acoustic echoes which are captured as a series of A-scans. These A-scans can then be combined to form a B-scan of a localized region of interest within the eye.

The UBM method is capable of making qualitative ultrasound images of the anterior segment of the eye but cannot make accurate, precision, comprehensive, measurable images of the cornea, lens or other components of the eye required for glaucoma screening, keratoconus evaluation or lens sizing. This is because of two reasons. First, the UBM is a hand-held device and relies on the steadiness of the operator's hand to maintain a fixed position relative to the eye being scanned for several seconds. Second, the UBM is pressed firmly onto the patient's eye to make contact with the patient's cornea to obtain good acoustic coupling. This gives rise to some distortion of the cornea and the eyeball.

Between these two limitations, the resolution is limited, at best, to the range of 40 to 60 microns and the reproducibility, at best, can be no better than 20 microns (“Ultrasound Biomicroscopy in Plateau Iris Syndrome”, Pavlin, Ritch and Foster, American Journal of Ophthalmology 113:390-395, April 1992 which is incorporated herein by reference).

Optical Coherence Tomography (OCT) is a light-based imaging technology that can image the cornea although not to the full lateral extent as can an ultrasound instrument. OCT cannot see behind the scleral wall or behind the iris and is therefore of limited use in glaucoma screening. OCT does well for imaging the central retina although only to the lateral extent allowed by a dilated pupil. OCT images of the retina can disclose the damage caused by glaucoma. The approach of a precision ultrasound scanning device is to detect the onset of glaucoma by imaging structural changes in the anterior segment before any retinal damage occurs so that the disease can be identified and successfully treated with drugs and/or stents.

The use of ultrasonic imaging of important features of the eye for lens implantation is discussed, for example, in U.S. Pat. No. 7,048,690. This patent does not include techniques for imaging the posterior surface of the lens capsule and so cannot be used to compute the volume of a lens capsule. Means for obtaining a full image of the lens capsule are disclosed in U.S. Pat. No. 8,317,709.

Other new procedures such as implantation of stents in or near the suprachoroid may provide part or all of a treatment for glaucoma. Ultrasonic imaging can be used to provide the required accurate images in the corner of the eye between the sclera and the iris (in the region of the suprachoroidal space to the scleral spur) which is well off-axis and essentially inaccessible to optical imaging.

In a normal human eye, intraocular pressure ranges from about 10 to about 22 mm Hg. An eye pressure of greater than about 22 mm Hg is considered higher than normal. An accepted average value of intraocular pressure is 15.5 mm Hg with fluctuations of about 2.75 mm Hg.

An ultrasonic scan of the eye may include one or more B-scans (each B-scan formed from a plurality of A-scans) and these may be combined automatically to form a comprehensive image of the anterior segment. Therefore it is necessary to rapidly scan a patient to reduce the possibility of patient eye motion during a scan session. Rapid scans can cause motion of the instrument as the transducer carriage and scan head move back and forth in the water bath.

Both ultrasound sector and ultrasound arc scanning instruments record time-of-arrival of reflected ultrasound pulses. A speed of sound of the medium is then used to convert these time of arrival measurements to distance measurements. Traditionally, a single representative speed of sound value is used. Usually the speed of sound of water at 37C (1,531 m/s) is used although speeds of sound from 1,531 m/s to 1,641 m/s may be used (1,641 m/s is the speed of sound in a natural human lens).

The speed of sound varies in the different anterior segment regions of the eye such as the cornea, aqueous, natural lens and vitreous fluid. The speed of sound in these different regions have been measured by various researchers and are reasonably known. Therefore if the interfaces of these regions can be identified, the appropriate speeds of sounds for these regions can be used to convert times of arrivals to distances with more accuracy.

It is also important to compensate for unintended patient head or eye motion because a scan of the anterior segment scan or lens capsule scan is typically made by overlaying two or three separate scans (such as an arcuate scan followed by two linear scans, also described in U.S. Pat. No. 9,597,059 entitled “Tracking Unintended Eye Movements in an Ultrasonic Scan of the Eye”.

Unintended patient eye motion includes saccades which are quick, simultaneous rotations of both eyes in the same direction involving a succession of discontinuous individual rotations of the eye orbit in the eye socket.

The speed of transducer motion in an precision scanning device such as described, for example, in U.S. Pat. No. 8,317,709, is limited because its movement is in a bath of water and excessive speed of motion of the transducer and its carriage can result in vibration of the entire instrument. In practice, a set of ultrasound scans can be carried out in about 1 to about 3 minutes from the time the patient's eye is immersed in water to the time the water is drained from the eyepiece.

The actual scanning process itself can be carried out in several tens of seconds, after the operator or automated software completes the process of centering and range finding. As is often the case, the patient may move his or her head slightly or may move his or her eye in its socket during this time. In some cases, the patient's heartbeat can be detected as a slight blurring of the images. If patient movements are large, the scan set can always be repeated.

The arc scanning instrument of the present disclosure can create several distinct scan types. These are:
an arcuate scan having a fixed radius of curvature
a linear scan
a combined arcuate and linear scan allowing for various radii of curvature including inverse radii of curvature

These scans can be combined to form composite images because each image is formed from very accurate time-of-arrival data and transducer positional data. However, combining these separate scans into a composite scan must take into account patient eye movement during scanning; and instrument movement during scanning.

Due to the need for an eye seal to provide a continuous medium for the ultrasound signal to travel between the transducer, any scanning device has a limitation in the range of movement the transducer can make relative to the eye. The range of the scanning device can be expanded to cover more of the anterior segment by introducing intentional and controlled eye movements and scanning the newly exposed portion of the eye that can now be reached. Registration techniques can be used to combine the scans of different eye positions to create a more complete composite image of the anterior segment of the eye.

U.S. patent application Ser. No. 16/422,182 entitled “Method for Measuring Behind the Iris after Locating the Scleral Spur” is pending. This application is directed towards a method for locating the scleral spur in an eye using a precision ultrasound scanning device for imaging of the anterior segment of the eye. One of the applications of a precision ultrasound scanning device or instrument is to image the region of the eye where the cornea, iris, sclera and ciliary muscle are all in close proximity. By using a knowledge of the structure of the eye in this region and employing binary filtering techniques, the position of the scleral spur can be determined. Once the position of the scleral spur is determined, a number of measurements that characterize the normal and abnormal shapes of components within this region of the anterior segment of the eye can be made. Many of the ideas disclosed in this application may be used to form accurate composite images.

Medications, lasers, and incisional surgery are used to lower the intraocular pressure to treat patients with glaucoma in order to prevent progressive optic nerve damage. One technique that is required at times is ‘ablation’ of the ciliary body—the portion within the eye that produces the aqueous humor. This procedure partially damages the ciliary body so that it makes less of the fluid that is responsible for the pressure in the eye. It tends to be saved for times when other surgical interventions have all failed. It used to be only for eyes that were almost completely blind but by titrating the dosage, it can be an option even if there is some vision intact. There is a related procedure called endoscopic cycloablation which is a more precisely delivered treatment but requires a visit to an operating room as it is performed by inserting a delivery probe into the eye.

Ciliary body ablation involves the injection of a drug into the vitreous chamber. The drugs used for injection are toxic to the ciliary body, which is the structure responsible for production of aqueous humor.

Cyclophotocoagulation is a laser treatment that targets the ciliary processes of the eye. The ciliary processes are the part of the eye that produce the fluid, or aqueous humor that bathes the tissues in the front of the eye.

High-intensity focused ultrasound (HIFU) is a non-invasive therapeutic technique that uses non-ionizing ultrasonic waves to heat tissue. HIFU can be used to increase the flow of blood or to destroy tissue, such as tumors, through a number of mechanisms. The technology is similar to ultrasonic imaging, although practiced at lower frequencies and higher acoustic power. Acoustic lenses may be used to achieve the necessary intensity at the target tissue without damaging the surrounding tissue. “Systematic Review of the Efficacy and Safety of Hgh-Intensity Focused Ultrasound for the Primary and Salvage Treatment of Prostate Cancer”, M. Warmuth, T. Johansson, P. Mad, European Urology 58 (2010) 803-815, Sep. 17, 2010.

A typical HIFU transducer has a diameter of about 19 mm with a center frequency of about 5 MHz, a focal length of about 15 mm and a focal intensity of about 200 W/mm². Another typical HIFU transducer has a diameter of about 60 mm with a center frequency of about 1 MHz, a focal length of about 75 mm and a focal intensity of about 17 W/mm².

Normal intraocular eye pressure ranges from about 12 to about 22 mm Hg. An intraocular eye pressure of greater than about 22 mm Hg is considered higher than normal. An average value of intraocular pressure is 15.5 mm Hg with fluctuations of about 2.75 mm Hg.

The external pressure on an eye can be increased above atmospheric pressure by raising or lowering the saline bag by an inch or two above the patient's eye. This pressure change can change the shape of the cornea or globe. Pressure on the outside of the eye can be changed by about 1 mm Hg for every half inch of saline bag elevation. (average IOP is ˜15.5 mm Hg).

In a normal human eye, intraocular pressure ranges from about 10 to about 22 mm Hg. An eye pressure of greater than about 22 mm Hg is considered higher than normal. An accepted average value of intraocular pressure is 15.5 mm Hg with fluctuations of about 2.75 mm Hg.

If IOP remains above normal for an extended period of time, glaucoma and damage to the retina can develop. There remains, therefore, a need for a method and apparatus to simultaneously image and treat a patient for high intraocular pressure without causing further damage to the retina.

### SUMMARY

The present disclosure is directed to a system for imaging and treating an eye of a patient that can include:

one or more ultrasound transducers;

a positioning mechanism that displaces the one or more ultrasound transducers into a desired location relative to an eye of the patient;

wherein:

in a first mode, the one or more ultrasound transducers emits ultrasound energy at a first range of frequencies to acquire an image of at least a portion of the eye of the patient; and in a second mode, the one or more ultrasound transducers emits ultrasound energy at a second range of frequencies to alter a physical characteristic of the eye, wherein the first range of frequencies is different than the second range of frequencies.

The present disclosure is directed to an ultrasound device for an eye of a patient, comprising:

an eyepiece positioned near the eye, the eyepiece having an interior volume; a window portion positioned on a surface of the eyepiece, the window portion being substantially parallel to the surface of the eye and substantially acoustically transparent, wherein the surface of the eyepiece is configured to operatively engage the eye of a patient;

a fluid disposed in the interior volume of the eyepiece;

a user interface to receive input from a user;

a processor;

a computer readable medium in communication with the processor; and

one or more ultrasound transducers positioned outside the fluid in the interior volume of the eyepiece, the one or more ultrasound transducers operably interconnected to at least one of an arcuate or linear track, wherein the instructions, when executed by the processor, cause the processor to operate in first and second modes:

in the first mode, the one or more ultrasound transducers emits ultrasound energy at a first range of frequencies to acquire an image of at least a portion of the eye of the patient; and in the second mode, the one or more ultrasound transducers emits ultrasound energy at a second range of frequencies to alter a physical characteristic of the eye, wherein the first range of frequencies is different than the second range of frequencies.

The present disclosure is directed to a method for imaging and treating an eye of a patient that can include the steps:

emitting, by one or more ultrasound transducers, ultrasound energy at a first range of wavelengths to acquire an image of at least a portion of an eye of a patient; and thereafter, emitting, by the one or more ultrasound transducers, ultrasound energy at a second range of wavelengths to alter a physical characteristic of the eye, wherein the first range of wavelengths is different than the second range of wavelengths.

In the first and second modes, the positioning mechanism can be in a common spatial location, the at least a portion of the eye can comprise a ciliary body and/or trabecular meshwork, and the physical characteristic of the eye can be one or more of an intraocular pressure, radius of a cornea, radius of a lens, cornea thickness, lens thickness, angle between peripheral edges of the lens and cornea, an angle between a back of the sclera and/or cornea and the front of the iris, on-axis distance between an anterior surface of the cornea and the anterior surface of the lens, on axis distance between a posterior surface of the cornea and the posterior surface of the lens, on axis distance between the posterior surface of the lens and the anterior surface of the retina, sclera parameter, and iris/pupil ratio. As will be appreciated, the physical characteristic can be any other ocular parameter that is a function, either directly or inversely, of the intraocular pressure.

In the first mode, the acquired image can comprise a plurality of A-scan images of the at least a portion of an eye of a patient, the processor can convert the plurality of A-scans to a plurality of B-scans, the plurality of B-scans can comprise images of the ciliary body and trabecular meshwork, and in the second mode, the one or more transducers can ablate at least a portion of the ciliary body and/or vibrate the trabecular mesh.

A mode frequency of the first range of frequencies can be different than a mode frequency of the second range of frequencies.

The processor can:

determine, from a first image of the at least a portion of the eye, a first set of measurements, the first image being acquired before second mode and from a second image of the at least a portion of the eye, a second set of measurements, the second image being acquired after the second mode; and

compare the first and second sets of measurements to determine a degree of alteration of the physical characteristic of the eye.

The physical characteristic of the eye can comprise an angle between peripheral edges of the lens and cornea and/or a dimension or angle of the sclera, wherein in the first and second modes the one or more transducers can have a commonly positioned focal point, wherein a median frequency of the first range of frequencies can be different than a median frequency of the second range of frequencies and wherein a mean frequency of the first range of frequencies can be different than a mean frequency of the second range of frequencies.

The second mode can include:

the one or more ultrasound transducers emitting ultrasound energy at the second range of frequencies to ablate a ciliary boy of the eye; and

the one or more ultrasound transducers vibrating a trabecular meshwork of the eye, wherein the second range of frequencies is different than the third range of frequencies.

To achieve high resolution images, coded excitation, tissue harmonic imaging, advanced transducers operating in the 35 MHz to 80 MHz range can be used to achieve a strong signal-to-noise reflection. The irradiating transducer can be, for example, about a 12 MHz transducer that would produce a strong second harmonic at about 24 MHz that could be used for imaging. The imaging transducer typically operates in the range of about 25MHz to about 40 MHz.

The following definitions are used herein:

The phrases at least one, one or more, and and/or are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together.

Acoustic impedance means the product of sound speed times density, pc, where p is the density (˜993 kg/cu meter for water at 37 C) and c is the sound speed (1,520 meters per second at 37 C). Thus acoustic impedance of water at 37 C is about 1.509×10⁶ kg/(sq meter-sec) or 1.509 Mrayls.

An acoustically reflective surface or interface is a surface or interface that has sufficient acoustic impedance difference across the interface to cause a measurable reflected acoustic signal. A specular surface is typically a very strong acoustically reflective surface.

The angle as referred to herein is the angle between the iris, which makes up the colored part of the eye, and the cornea, which is the clear-window front part of the eye. The angle is short for the iridocorneal angle. When the angle is open, most, if not all, of the eye's drainage system can be seen by using a special mirrored lens. When the angle is narrow, only portions of the drainage angle are visible, and in acute angle-closure glaucoma, none of it is visible. The angle is the location where the fluid that is produced inside the eye, the aqueous humor, drains out of the eye into the body's circulatory system. The function of the aqueous humor is to provide nutrition to the eye and to maintain the eye in a pressurized state. Aqueous humor should not be confused with tears since aqueous humor is inside the eye.

Anterior means situated at the front part of a structure; anterior is the opposite of posterior.

The anterior segment is the front third of the eye that includes the structures in front of the vitreous humor: the cornea, iris, ciliary body, and lens. Within the anterior segment are two fluid-filled spaces: the anterior chamber between the posterior surface of the cornea (i.e. the corneal endothelium) and the iris and the posterior chamber between the iris and the front face of the vitreous. Aqueous humor fills these spaces within the anterior segment and provides nutrients to the surrounding structures.

An A-scan is a representation of a rectified, filtered reflected acoustic signal as a function of time, received by an ultrasonic transducer from acoustic pulses originally emitted by the ultrasonic transducer from a known fixed position relative to a body component.

Accuracy as used herein means substantially free from measurement error.

An arc scanner, as used herein, is an ultrasound eye scanning device where the ultrasound transducer moves back and forth along an arcuate guide track wherein the focal point of the ultrasound transducer is typically placed somewhere within the eye near the region of interest (i.e. the corneas, the lens etcetera). The scanner may also include a linear guide track which can move the arcuate guide track laterally such that the effective radius of curvature of the arcuate track is either increased or decreased. The scanner utilizes a transducer that both sends and receives pulses as it moves along 1) an arcuate guide track, which guide track has a center of curvature whose position can be moved to scan different curved surfaces; 2) a linear guide track; and 3) a combination of linear and arcuate guide tracks which can create a range of centers of curvature whose position can be moved to scan different curved surfaces.

Automatic refers to any process or operation done without material human input when the process or operation is performed. However, a process or operation can be automatic, even though performance of the process or operation uses material or immaterial human input, if the input is received before performance of the process or operation. Human input is deemed to be material if such input influences how the process or operation will be performed. Human input that consents to the performance of the process or operation is not deemed to be “material.”

Body habitus is somewhat redundant, since habitus by itself means “physique or body build.”. Body size and habitus describe the physical characteristics of an individual and include such considerations as physique, general bearing, and body build.

A B-scan is a processed representation of A-scan data by either or both of converting it from a time to a distance using acoustic velocities and by using grayscales, which correspond to A-scan amplitudes, to highlight the features along the A-scan time history trace (the latter also referred to as an A-scan vector).

The ciliary body is a circular structure that is an extension of the iris, the colored part of the eye. The epithelium of the ciliary body produces the fluid in the eye called aqueous humor (AH). It also contains the ciliary muscle, which changes the shape of the lens when your eyes focus on a near object. This process is called accommodation. The ciliary body is the circumferential tissue inside the eye composed of the ciliary muscle and ciliary processes. There are three sets of ciliary muscles in the eye, the longitudinal, radial, and circular muscles. They are near the front of the eye, above and below the lens. They are attached to the lens by connective tissue called the zonule of Zinn, and are responsible for shaping the lens to focus light on the retina. When the ciliary muscle relaxes, it flattens the lens, generally improving the focus for farther objects. When it contracts, the lens becomes more convex, generally improving the focus for closer objects.

Coded excitations are engineered excitation pulses that are capable of increasing the effective penetration depth of a transmitted signal in echo location imaging systems such as radar, sonar and ultrasound, by improving the signal-to-noise ratio (SNR).

Chirping is a coded excitation that can be thought of as a complex exponential sequence with linearly increasing frequency. A linear chirp is a coded signal that linearly spans a frequency bandwidth B=f₂−f₁, where f₁ and f₂ are the starting and ending frequencies, respectively. If the chirp sweeps from f₁ to f₂ over a time, then the chirp-coded excitation is described by: s(t)=ω(t) cos(2πfit+πbt²).

Fiducial means a reference, marker or datum in the field of view of an imaging device.

Fixation means having the patient focus an eye on an optical target such that the eye's optical axis is in a known spatial relationship with the optical target. In fixation, the light source is axially aligned in the arc plane with the light source in the center of the arc so as to obtain maximum signal strength such that moving away from the center of the arc in either direction results in signal strength diminishing equally in either direction away from the center.

Glaucoma is an eye condition that damages the optic nerve. This damage is often caused by an abnormally high pressure in the eye. Glaucoma is one of the leading causes of blindness in older adults. Because vision loss due to glaucoma can't be recovered, it's important to have regular eye exams that include measurements of your eye pressure so a diagnosis can be made in its early stages and treated appropriately.

Hand-held ultrasonic scanner See Ultrasound Bio Microscopy (UBM).

HIFU means High-Intensity Focused Ultrasound.

An iatrogenic risk is a risk due to the activity of a physician or surgeon or by medical treatment or diagnostic procedures. For example, an iatrogenic illness is an illness that is caused by a medication or physician.

An imaging ultrasound transducer is the device that is responsible for creating the outgoing ultrasound pulse and detecting the reflected ultrasound signal that is used for creating the A-Scans and B-Scans.

“Intraocular pressure” refers to the fluid pressure inside the eye. Intraocular pressure is determined by the production and drainage of aqueous humor by the ciliary body and its drainage via the trabecular meshwork and uveoscleral outflow. The reason for this is because the vitreous humor in the posterior segment has a relatively fixed volume and thus does not affect intraocular pressure regulation. Current consensus among ophthalmologists and optometrists defines normal intraocular pressure as that between about 10 mm Hg and 20 mmHg.

As used herein, a meridian is a 2-dimensional plane section through the approximate center of a 3-dimensional eye and its angle is commonly expressed relative to a horizon defined by the nasal canthus and temporal canthus of the eye.

Positioner means the mechanism that positions a scan head relative to a selected part of an eye. In the present disclosure, the positioner can move back and forth along the x, y or z axes and rotate in the β direction about the z-axis. Normally the positioner does not move during a scan, only the scan head moves. In certain operations, such as measuring the thickness of a region, the positioner may move during a scan.

Position tracking sensors are a set of position sensors whose sole purpose is to monitor the movement of the eye or any other anatomical feature during the imaging scan so as to remove unwanted movement of the feature.

Posterior means situated at the back part of a structure; posterior is the opposite of anterior.

Precise as used herein means sharply defined and repeatable.

Precision means how close in value successive measurements fall when attempting to repeat the same measurement between two detectable features in the image field. In a normal distribution precision is characterized by the standard deviation of the set of repeated measurements. Precision is very similar to the definition of repeatability.

The pulse transit time across a region of the eye is the time it takes a sound pulse to traverse the region.

A pulser/receiver board carries the electronics required 1) to shape the electrical pulse to drive the ultrasound transducer; 2) to receive the return signal from the ultrasound pulse engaging the target; and 3) to isolate the stronger driver pulse from the much weaker received pulse.

Refractive means anything pertaining to the focusing of light rays by the various components of the eye, principally the cornea and lens.

Scan head means the mechanism that comprises the ultrasound transducer, the transducer holder and carriage as well as any guide tracks that allow the transducer to be moved relative to the positioner. Guide tracks may be linear, arcuate or any other appropriate geometry. The guide tracks may be rigid or flexible. Normally, only the scan head is moved during a scan.

Schlemm's canal is a circular lymphatic-like vessel in the eye that collects aqueous humor from the anterior chamber and delivers it into the episcleral blood vessels via aqueous veins. The canal is essentially an endothelium-lined tube, resembling that of a lymphatic vessel. On the inside of the canal, nearest to the aqueous humor, it is covered by the trabecular meshwork; this region makes the greatest contribution to outflow resistance of the aqueous humor. The molecular identity of Schlemm's canal is very similar to the one of lymphatic vasculature.

The “sclera” also known as the “white of the eye” or, in older literature, as the “tunica albuginea oculi”, is the opaque, fibrous, protective, outer layer of the human eye containing mainly collagen and some crucial elastic fiber.

Sector scanner is an ultrasonic scanner that sweeps a sector like a radar. The swept area is pie-shaped with its central point typically located near the face of the ultrasound transducer.

A specular surface means a mirror-like surface that reflects either optical or acoustic waves. For example, an ultrasound beam emanating from a transducer will be reflected directly back to that transducer when the beam is aligned perpendicular to a specular surface.

Tissue means an aggregate of cells usually of a particular kind together with their intercellular substance that form one of the structural materials of a plant or an animal and that in animals include connective tissue, epithelium, muscle tissue, and nerve tissue.

A track or guide track is an apparatus along which another apparatus moves. In an ultrasound scanner or combined ultrasound and optical scanner, a guide track is an apparatus along which one or more ultrasound transducers and/or optical probes moves during a scan.

Tissue harmonic imaging exploits non-linear propagation of ultrasound through the body tissues. The high pressure portion of the wave travels faster than low pressure resulting in distortion of the shape of the wave. This change in waveform leads to generation of harmonics (multiples of the fundamental or transmitted frequency) from tissue. Typically, the 2nd harmonic is used to produce the image as the subsequent harmonics are of decreasing amplitude and hence insufficient to generate a proper image. These harmonic waves that are generated within the tissue increase with depth to a point of maximum intensity and then decrease with further depth due to attenuation. Hence the maximum intensity is achieved at an optimum depth below the surface. Advantages over conventional ultrasound include: decreased reverberation and side lobe artifacts; increased axial and lateral resolution; increased signal to noise ratio; and improved resolution in patients with large body habitus.

The angle of opening, called the trabecular-iris angle (TIA), is defined as an angle measured with the apex in the iris recess and the arms of the angle passing through a point on the trabecular meshwork 500 μm from the scleral spur and the point on the iris perpendicularly. The TIA is a specific way to measure the angle or iridocorneal angle.

The trabecular meshwork (TM) is a lamellated sheet of complex tissue that covers the inner wall of Schlemm's canal. TM has uniquely developed at the angle of primates, filtering the aqueous humor out of the eye. TM consists of two parts: the nonfiltering portion mainly occupied by trabecular cells and the filtering portion. Trabecular cells are highly phagocytic cells removing particles, cell debris, and protein from the aqueous humor. The first glaucoma locus, the trabecular meshwork inducible glucocorticoid response (TIGR), also known as myocilin, initially was identified by looking at genes whose transcription is highly induced by steroids in these cells. The filtering portion consists of three tissues: the cribriform layer, the corneoscleral meshwork, and the uveal meshwork. These trabecular beams or strands are intertwiningly connected to each other, forming a complex filtering mesh surrounding Schlemm's canal. The trabecular beams are thickened by accumulation of extracellular materials and decrease of cell density within the corneoscleral and uveal meshwork in aged eyes.

Ultrasonic or ultrasound means sound that is above the human ear's upper frequency limit. When used for imaging an object like the eye, the sound passes through a liquid medium, and its frequency is many orders of magnitude greater than can be detected by the human ear. For high-resolution acoustic imaging in the eye, the frequency is typically in the approximate range of about 5 to about 80 MHz.

Ultrasound Bio Microscopy (UBM) is an imaging technique using hand-held ultrasound device that can capture anterior segment images using a transducer to emit short acoustic pulses ranging from about 20 to about 80 MHz. This type of ultrasound scanner is also called a sector scanner. The UBM method is capable of making qualitative ultrasound images of the anterior segment of the eye but cannot unambiguously make accurate, precision, comprehensive, measurable images of the cornea, lens or other components of the eye.

A vector refers to a single acoustic pulse and its multiple reflections from various eye components. An A-scan is a representation of this data whose amplitude is typically rectified.

Water equivalent as used herein means a fluid or a gel having the approximate acoustic impedance of water.

### BRIEF DESCRIPTION OF THE DRAWINGS

The present disclosure may take form in various components and arrangements of components, and in various steps and arrangements of steps. The drawings are only for purposes of illustrating the preferred embodiments and are not to be construed as limiting the disclosure. In the drawings, like reference numerals may refer to like or analogous components throughout the several views.

FIG. 1 is a prior art schematic of a human eye illustrating the ciliary body and trabecular mesh.

FIG. 2 is a prior art schematic of a human eye illustrating the flow of aqueous fluid from the ciliary body to the trabecular mesh.

FIG. 3 is another prior art schematic of a human eye illustrating the flow of aqueous fluid from the ciliary body to the trabecular mesh.

FIG. 4 is yet another prior art schematic of a human eye illustrating the flow of aqueous fluid from the ciliary body to the trabecular mesh.

FIG. 5A illustrates features of the human eye.

FIG. 5B illustrates typical dimensions of features of the human eye.

FIG. 6 is a schematic of an arc scanner for imaging an eye using ultrasound in accordance with an embodiment of the disclosure.

FIG. 7A is a schematic of a prior art single element ultrasound transducer assembly.

FIG. 7B is a schematic of a prior art dual element ultrasound transducer assembly.

FIG. 8 is a schematic of the transducer face of a prior art multi-element ultrasound transducer configuration.

FIGS. 9A and 9B are examples of simple emitted and received ultrasound pulse waveforms.

FIG. 10 shows examples of several known emitted chirp ultrasound pulse waveforms.

FIG. 11 is a schematic of an arc scanning device according to an embodiment of the disclosure.

FIG. 12 is a schematic of an arc scanning device with multiple probes according to an embodiment of the disclosure.

FIG. 13 is a graph of intraocular pressure (IOP) of a human eye in millimeters of mercury versus water pressure in inches of water column height.

FIG. 14 is a graph of pressure differential across the cornea versus IOP.

FIG. 15 is a schematic representation of an arc scanning system according to an embodiment of the present disclosure.

FIG. 16 depicts the control and signal processing elements for any of the embodiments of the present disclosure.

FIGS. 17A and 17B provide a sequence of operations according to embodiments of this disclosure.

FIGS. 18A and B provide a sequence of summarized intraocular pressures (measured in mm Hg) in various regions of the eye during an imaging and treatment process according to an embodiment of this disclosure.

### DETAILED DESCRIPTION

The disclosure is directed to a method for treating a human eye having elevated intraocular pressure (potentially caused by ocular hypertension or glaucoma) that includes imaging the anterior segment of an eye over a first range of ultrasound frequencies and amplitudes; then ablating the ciliary body over a different second range of ultrasound frequencies and amplitudes; and/or vibrating the trabecular mesh over a different third range of ultrasound frequencies and amplitudes.

In this disclosure, high intensity ultrasound energy is proposed to ablate the ciliary body rather than cyclophotocoagulation. High intensity ultrasound energy is known to be effective in ablating tissue in the treatment of the prostate gland. It is expected that high intensity ultrasound energy will also partially ablate the ciliary body so that it makes less of the fluid that is responsible for higher pressures in the eye causing cyclophotocoagulation.

Cyclophotocoagulation has a risk of damaging the retina whereas high intensity ultrasound energy is strongly attenuated as it propagates through the approximately 16 mm of vitreous humor to reach the retina.

The apparatus of the present disclosure can include both an imaging transducer and higher powered ultrasound irradiating transducer. Both are mounted on the same carriage as part of a scan head. The scan head is positioned with respect to a patient's eye using a positioner mechanism. The scan head may include a probe carriage for moving the imaging and irradiating probes. The positioner mechanism, the scan head, and probe carriage may be immersed in water. A disposable eyepiece may be connected to the system and filled separately with water to provide a continuous water transmission path from the probes to the surface of patient's eye.

The probe carriage can comprise an imaging ultrasound transducer probe, an irradiating ultrasound transducer, and a third transducer holder. The ultrasound imaging transducer probe and ultrasound irradiating probe are preferably focused at the same point on or within the patient's eye. Alternately or additionally, the probes can be substantially parallel and then offset by a small linear dimension.

In a preferred mode, the imaging transducer and the irradiating therapeutic transducer may be mounted on a revolver type holder. The imaging transducer may be rotated about a rotational axis into position with respect to the eye and an image made of the eye. Then the irradiating transducer may be rotated into position about the same rotational axis with respect to the eye. Then the imaging transducer may be rotated back into position about the common rotational axis and an image made of the irradiated eye. In this way, the imaging transducer and the irradiating therapeutic transducer are in the same position when emitting ultrasound energy into the eye.

In another mode, a single irradiating therapeutic transducer can be used in a conventional holder. The irradiating therapeutic transducer required is typically in the range of about 17 mm diameter to about 30 mm in diameter, typically in the frequency range of about 5 MHz to about 20 MHz and typically has a focal length in the range of about 20 mm to about 40 mm. When operating in this second mode, coded excitation and tissue harmonic imaging techniques may be used to image the irradiated tissue. For example, a 15 MHz irradiating transducer would produce a strong second harmonic at about 30 MHz that could be used for imaging.

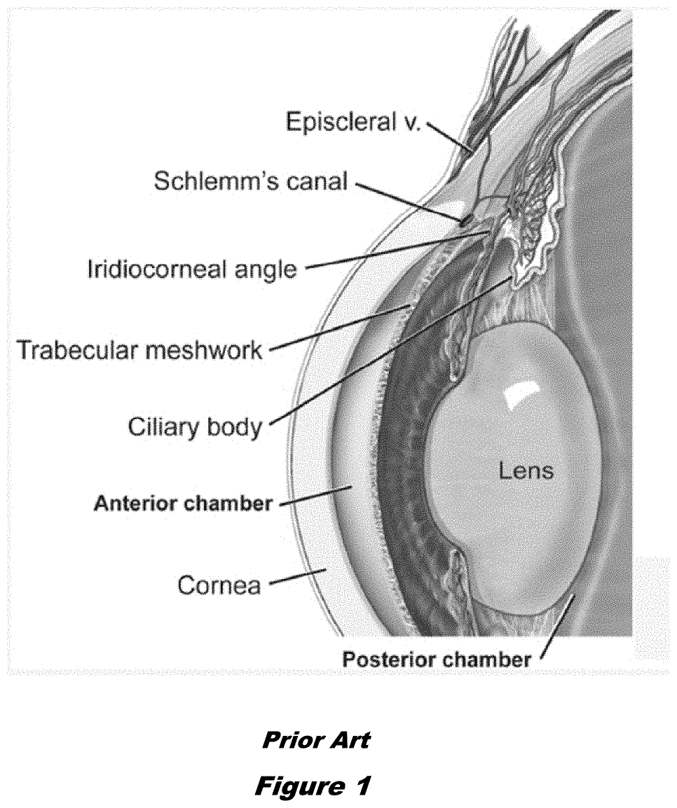

FIG. 1 is a prior art schematic of a human eye illustrating the ciliary body and trabecular mesh. The ciliary body is a circular structure that is an extension of the iris, the colored part of the eye. The ciliary body produces the fluid in the eye called aqueous humor. It also contains the ciliary muscle, which changes the shape of the lens when the eye focuses on a near object. This process is called accommodation. The ciliary body is the circumferential tissue inside the eye composed of the ciliary muscle and ciliary processes. There are three sets of ciliary muscles in the eye, the longitudinal, radial, and circular muscles. They are near the front of the eye, above and below the lens. They are attached to the lens by connective tissue called the zonule or Zinn, and are responsible for shaping the lens to focus light on the retina. When the ciliary muscle relaxes, it flattens the lens, generally improving the focus for farther objects. When it contracts, the lens becomes more convex, generally improving the focus on closer objects.

The trabecular meshwork is a lamellated sheet of complex tissue that covers the inner wall of Schlemm's canal. Schlemm's canal is a circular lymphatic-like vessel in the eye that collects aqueous humor from the anterior chamber and delivers it into the episcleral blood vessels via aqueous veins. The canal is essentially an endothelium-lined tube, resembling that of a lymphatic vessel. On the inside of the canal, nearest to the aqueous humor, it is covered by the trabecular meshwork; this region makes the greatest contribution to outflow resistance of the aqueous humor.

The angle of opening, called the trabecular-iris angle (TIA), is defined as an angle measured with the apex in the iris recess and the arms of the angle passing through a point on the trabecular meshwork 500 μm from the scleral spur and the point on the iris perpendicularly. The TIA is a specific way to measure the angle or iridocorneal angle.

The trabecular mesh has uniquely developed at the angle in primates, filtering the aqueous humor out of the eye. The trabecular mesh consists of two parts: the nonfiltering portion mainly occupied by trabecular cells and the filtering portion. Trabecular cells are highly phagocytic cells removing particles, cell debris, and protein from the aqueous humor. The first glaucoma locus, the trabecular meshwork inducible glucocorticoid response (TIGR), also known as myocilin, initially was identified by looking at genes whose transcription is highly induced by steroids in these cells. The filtering portion consists of three tissues: the cribriform layer, the corneoscleral meshwork, and the uveal meshwork. These trabecular beams or strands are intertwiningly connected to each other, forming a complex filtering mesh surrounding Schlemm's canal. The trabecular beams are thickened by accumulation of extracellular materials and decrease of cell density within the corneoscleral and uveal meshwork in aged eyes.

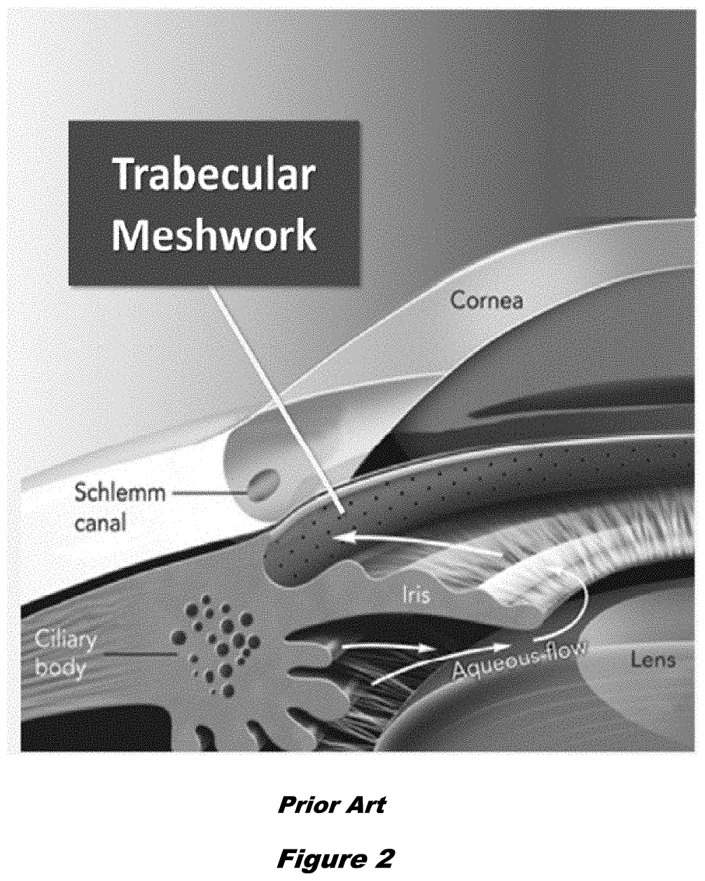

FIG. 2 is a prior art schematic of a human eye illustrating the flow of aqueous fluid from the ciliary body to the trabecular mesh. This illustration clearly shows the path of aqueous flow from the ciliary body, around the iris and through the trabecular mesh to Schlemm's canal.

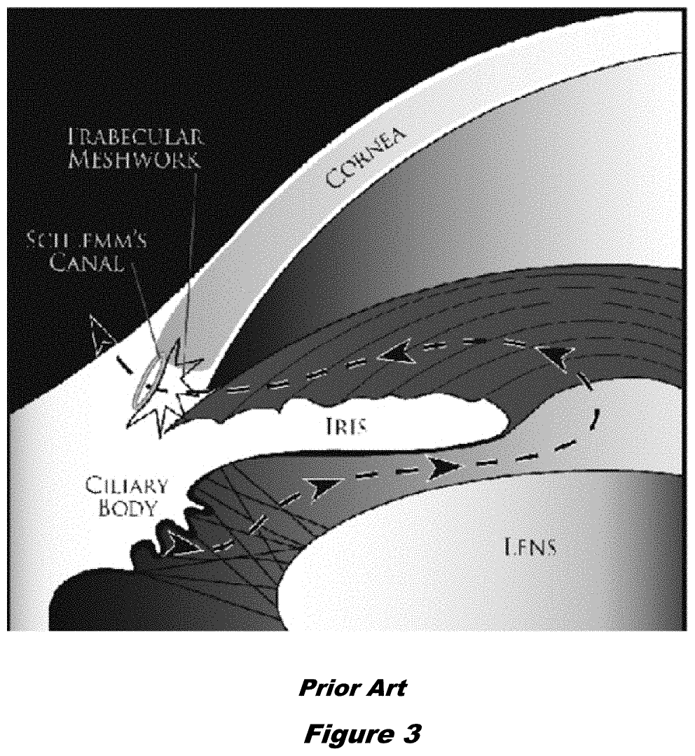

FIG. 3 is another prior art schematic of a human eye illustrating the flow of aqueous fluid from the ciliary body to the trabecular mesh.

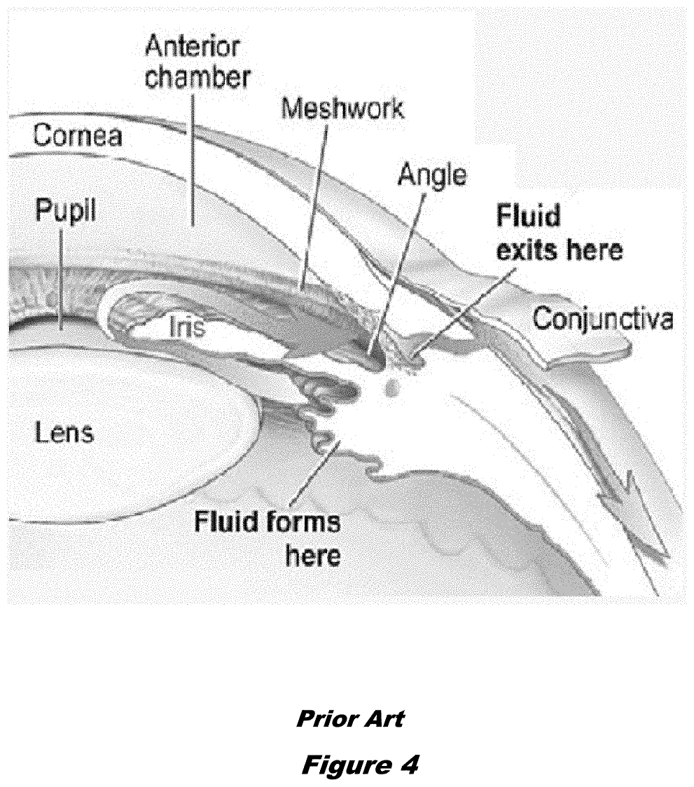

FIG. 4 is yet another prior art schematic of a human eye illustrating the region wherein aqueous fluid originates in the ciliary body and the region wherein aqueous fluid exits the anterior segment through the trabecular mesh.

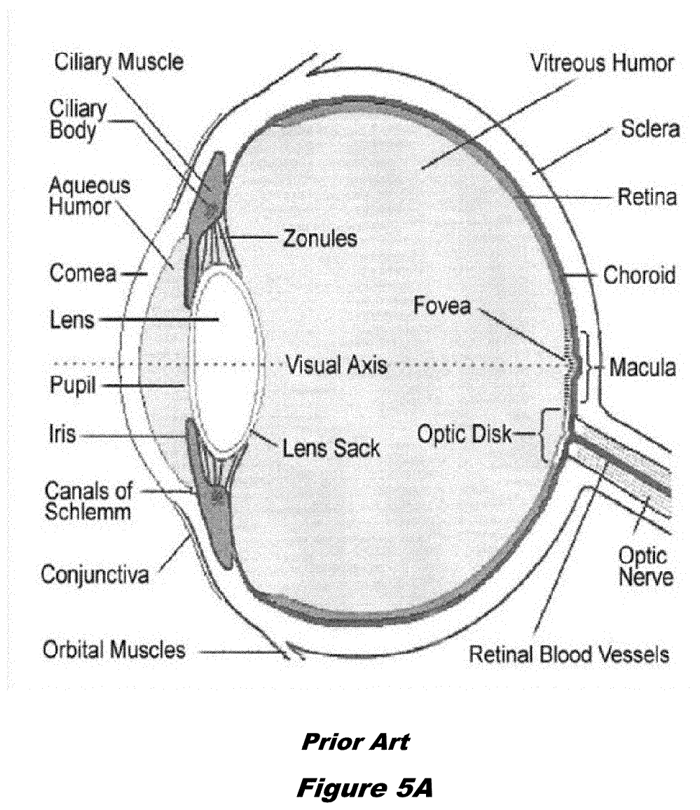

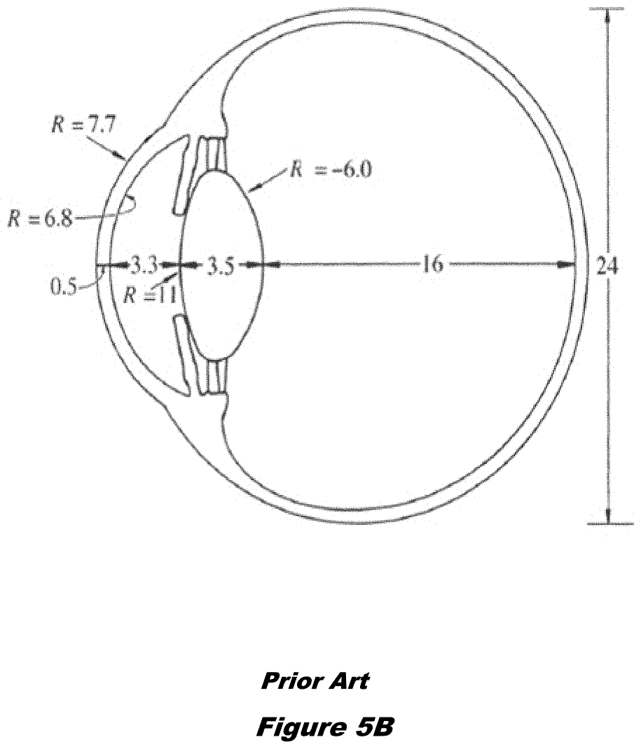

FIG. 5A is a prior art schematic that illustrates the main features of the human eye while FIG. 5B is a prior art schematic that illustrates the typical dimensions of the human eye (with the measurements in mm and “R”: refers to the radius). The anterior segment is the front third of the eye that includes the structures from the anterior surface of the cornea to the posterior surface of the lens.

An ultrasound scanning apparatus, such as described for example, in U.S. Pat. Nos. 8,317,709, 8,510,883, 8,317,702, and 8,758,252 is comprised of a positioning mechanism and a scan head. The positioning mechanism has x, y, z and beta (rotation about its z-axis) positioning mechanisms which make it possible to position the scan head relative to the eye component of interest. This operation is carried out while the patient's eye is positioned in contact with an eyepiece attached to the scanner and while the patient's head is fixed relative to the scanner by a head rest or by the eyepiece or by a combination of both. Once the positioning mechanism is set, the only moving part relative to the eye component of interest is the scan head. The scan head may be comprised of only an arcuate guide track which is typically used to produce an ultrasound scan of the cornea and/or much of the anterior segment of an eye. The scan head may be comprised of only a linear guide track. In another embodiment, the scan head may be comprised of an arcuate guide track and a linear guide track that can be moved in a combination of linear and arcuate motions to produce an ultrasound scan of the entire anterior segment including much of the posterior surface of the lens. The movement of the positioner and scan head relative to patient's eye socket is precisely known at all times by a system of magnetic encoder strips.

The movement of the scan head relative to the eye component of interest is therefore known with precision and accuracy as long as the patient does not move their eye during the scan. A single scan can take less than a second. A sequence of scans can take several seconds. A patient's eye can move significantly even during a single scan, thus degrading the precision and accuracy of the scan. The usual procedure, when this occurs, is to re-scan the patient. In US Publication No. 20130310692 entitled “Correcting for Unintended Motion for Ultrasonic Eye Scans”, a device and method of tracking any movement of the patient's eye, relative to the positioning mechanism, during a scan is described.

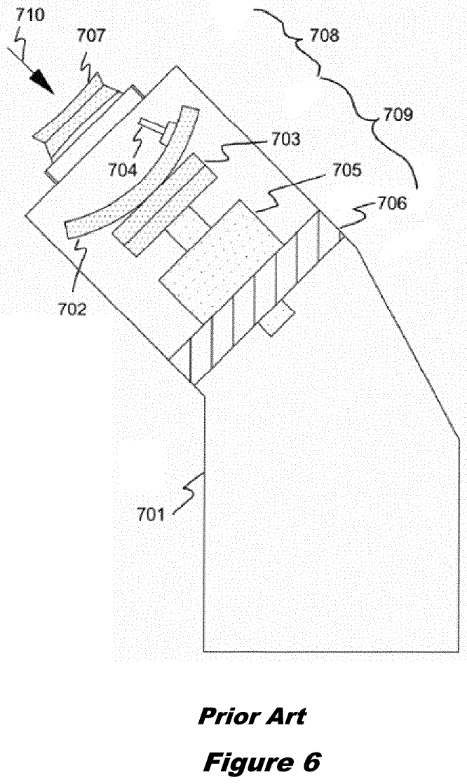

FIG. 6 is a schematic of the principal elements of an ultrasound eye scanning device. The scanning apparatus 701 of this example is comprised of a scan head assembly 708 (shown here as an arcuate guide 702 with scanning and irradiating therapeutic transducer 704 on a transducer carriage which moves back and forth along the arcuate guide track, and a linear guide track 703 which moves the arcuate guide track back and forth as described above), a positioning mechanism 709 comprised of an x-y-z and beta mechanisms 705 mounted on a base 706 which is rigidly attached to scanning apparatus 701, and a disposable eyepiece 707. The scanning machine 701 is typically connected to a computer (not shown) which includes a processor module, a memory module, and a video monitor. The patient is seated at the machine 701 with their eye engaged with disposable eyepiece 707. The patient is typically looking downward during a scan sequence. The patient is fixed with respect to the scanning machine 701 by a headrest system and by the eyepiece 707. The operator using, for example, a mouse and/or a keyboard and video screen inputs information into the computer selecting the type of scan and scan configurations as well as the desired type of output analyses. The operator, for example, again using a mouse and/or a keyboard, a video camera located in the scanning machine and video screen, then centers a reference marker such as, for example, a set of cross hairs displayed on a video screen on the desired component of the patient's eye to be imaged and/or therapeutically irradiated which is also displayed on video screen. This is done by setting one of the cross hairs as the prime meridian for scanning. These steps are carried out using the positioning mechanism which can move the scan head in the x, x, z and beta space (three translational motions plus rotation about the z-axis). Once this is accomplished, the operator instructs the computer using either a mouse and/or a keyboard to proceed with the scanning and/or therapeutic irradiating sequence. Now the computer processor takes over the procedure and issues instructions to the scan head 708 and the transducer 704 and receives positional and imaging data. The computer processor proceeds with a sequence of operations for imaging such as, for example: (1) with the transducer carriage substantially centered on the arcuate guide track, rough focusing of transducer 704 on a selected eye component; (2) accurately centering of the arcuate guide track with respect to the selected eye component; (3) accurately focusing transducer 704 on the selected feature of the selected eye component; (4) rotating the scan head through a substantial angle (including orthogonal) and repeating steps (1) through (3) on a second meridian; (5) rotating the scan head back to the prime meridian; (6) initiating a set of A-scans along each of the of selected scan meridians, storing this information in the memory module; (7) utilizing the processor, converting the A-scans for each meridian into a set of B-scans and then processing the B-scans to form an image associated with each meridian; (8) performing the selected analyses on the A-scans, B-scans and images associated with each or all of the meridians scanned; and (9) outputting the data in a preselected format to an output device such as storage disk drive or a printer. In therapeutic irradiation, the computer processor irradiates the desired component of the eye followed by additional imaging to determine new dimensions of the desired component of the eye (e.g., the ciliary body). The processor can then compare the new dimensions with the previously imaged dimensions and correlate the changes in dimension, shape, area, and/or volume of the desired component (e.g., the shape of the ciliary body, the radii of curvature of the anterior and posterior cornea and lens, the changes in the cornea and lens thicknesses, and/or the changes in the on-axis distances between the anterior surface of the cornea to the anterior surface of the lens and between the posterior surface of the lens to the anterior surface of the lens). These changes permit the processor to determine the pressure differential across the cornea and the reduction in IOP compared to the pre-irradiation or starting IOP. As will be appreciated, measured values of intraocular pressure are influenced by corneal thickness and rigidity (e.g., central corneal thickness). Alternatively or additionally, the IOP is measured after each treatment stage by tonometry techniques, including without limitation applanation tonometry, goldmann tonometry, perkins tonometry, dynamic contour tonometry, electronic indentation tonometry, rebound tonometry, pneumatonemetry, impression tonometry, non-corneal and transpalpebral tonometry, non-contact tonometry, an ocular response analyzer, and/or palpitation. As can be appreciated, the patient's head must remain fixed with respect to the scanning machine 601 during the above operations when scanning is being carried out, which in a modern ultrasound scanning machine can take several tens of seconds.

An eyepiece serves to complete a continuous acoustic path for ultrasonic scanning, that path extending in water from the transducer to the surface of the patient's eye. The eyepiece 707 also separates the water in which the patient's eye is immersed from the water in the chamber in which the transducer guide track assemblies are contained. The patient sits at the machine and looks down through the eyepiece 707 as shown by arrow 710. Finally, the eyepiece provides an additional steady rest for the patient and helps the patient to remain steady during a scan procedure.

As can be appreciated, the arcuate guide track used to image the eye has a radius of curvature similar to that of the cornea and anterior surface of the natural lens. If an arcuate guide track is used for imaging a prostate, for example, the radius of curvature can be appropriately adjusted by a combination of arcuate and linear motions such as described for example in U.S. Pat. No. 8,317,709. As can be further appreciated, the guide track can have another shape than arcuate or could, in principle, be made to flex in a precise way so as to custom fit a patient.

Annular Array Transducers, Coded Excitation and Tissue Harmonic Imaging Tissue harmonic imaging enables ultrasound images with higher signal-to-noise ratio and higher spatial resolution. Tissue harmonic imaging and coded excitation together can be applied wherein coded excitation can overcome the trade-off between spatial resolution and penetration, which occurs when using a conventional transmitted pulse. For example, a chirp signal is frequently used for medical ultrasound imaging. A chirp is a in which the frequency increases (up-chirp) or decreases (down-chirp) with time. A combination of coded excitation and tissue harmonic imaging has been found to produce superior ultrasound images.

The techniques of tissue harmonic imaging and coded excitation (chirped waveforms) can also be applied to higher amplitude ultrasonic beams to enable these beams to ablate the ciliary body and to vibrate the trabecular mesh to stimulate better throughput which will tend to reduce intraocular pressure.

### Single Element Ultrasound Transducer

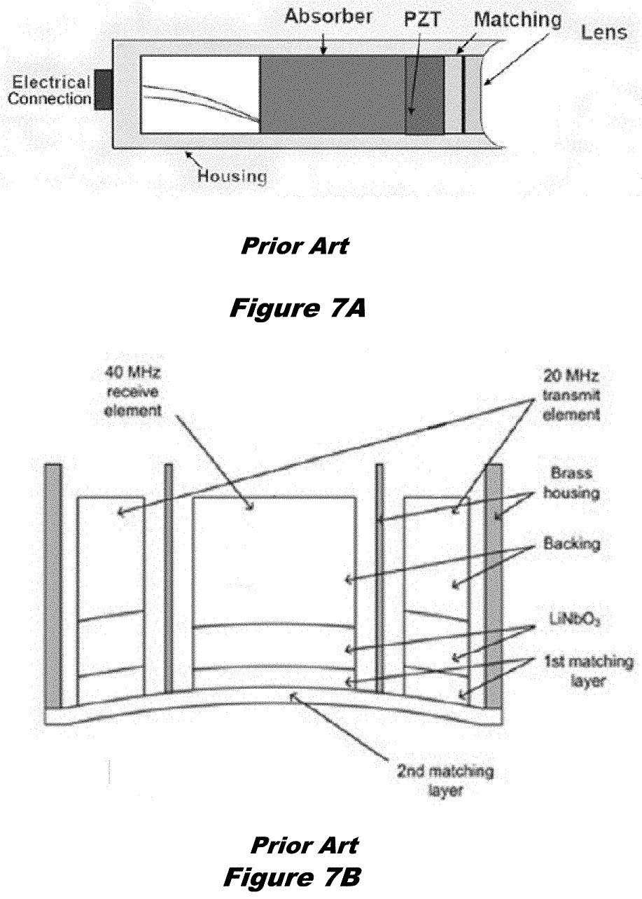

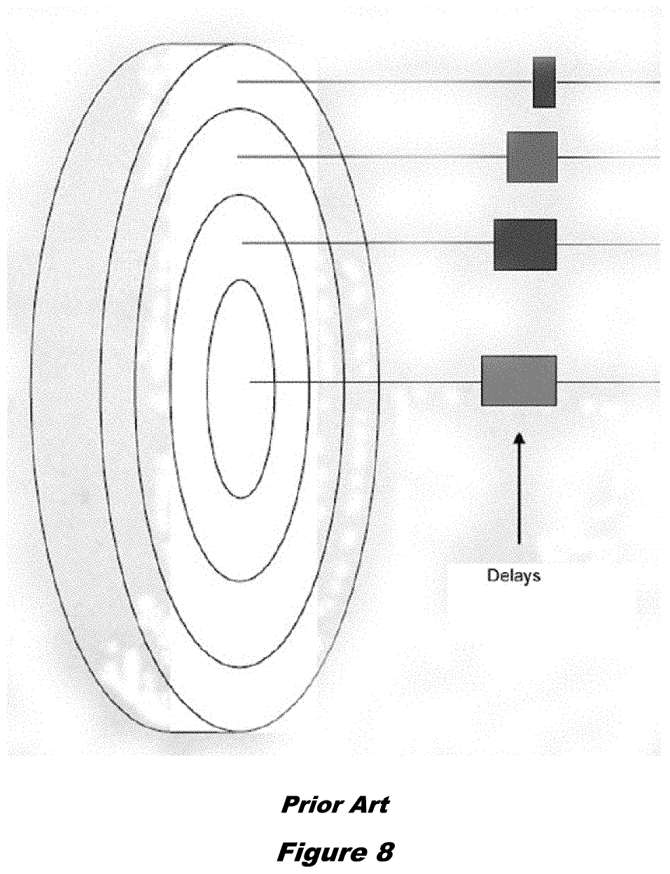

A prior art single element or needle transducer is shown in FIG. 7A (taken from a slide show entitled “Ultrasound Transducers” by Ravi Managuli). This type of transducer can be used in precision arc scanner of FIG. 6 discussed in the previous section. The single transducer element both transmits an ultrasound pulse and receives the echoed pulse.

### Annular Array Ultrasound Transducers

FIG. 7B is a schematic of a prior art dual element ultrasound transducer assembly as described in “20 MHz/40 MHz Dual Element Transducers for High Frequency Harmonic Imaging”, Kim, Cannata, Liu, Chang, Silverman and Shung, IEEE Transactions on Ultrasonics, Ferroelectrics and Frequency Control, \'OL. 55; NO. 12, December 2008. This type of transducer can also be used in precision arc scanner of FIG. 6 discussed in the previous section. Both the annular element and the center element can transmit and receive ultrasound pulses independently. In another mode, the center element can transmit and receive a pulse at one frequency while the annular element can transmit and receive a separate pulse at a different frequency. In yet another mode, the annular element can transmit and receive a pulse at one frequency while the center element can receive the echoed pulse at a harmonic frequency of the transmitted pulse.

As discussed in the above reference, a concentric annular type dual element transducer was used for second harmonic imaging to improve spatial resolution and depth of penetration for ophthalmic imaging applications. The outer ring element was designed to transmit a 20 MHz signal and the inner circular element was designed to receive the 40 MHz second harmonic signal.

Tissue harmonic ultrasound imaging has been accepted as one of the standard imaging modalities in many applications since its introduction to medical ultrasound imaging in the 1990s. Especially in cardiac and abdominal studies, tissue harmonic imaging is very often used for diagnostics along with fundamental imaging. By utilizing the second harmonic component of the received signal, images can be improved by reducing near field reverberation, decreasing phase aberration error, and improving border delineation.

In ophthalmology, imaging of the posterior segment which includes the retina, require improved spatial resolution and depth of penetration for proper diagnosis of retinal disease. This same second harmonic imaging technique can be used to improve imaging of, for example, the prostate.

Recently, broad band single element transducers operating at about 20 MHz have been used for imaging the posterior segment of the eye, but were limited in spatial resolution at that frequency. Unfortunately, transducers operating at 20 MHz cannot provide the spatial resolution needed to adequately delineate layers on the posterior segment of the human eye. Those operating in the higher frequency range do not provide sufficient depth of penetration such that the reflected signal can be detected above the noise floor. A concentric annular type dual element transducer for second harmonic imaging of the posterior segment of the eye wherein the outer ring element is used for transmit and the inner circular element for receive. A ring-shaped outer element produces higher side lobes than does a circular element of the same diameter, but this is to some degree compensated for by inherently lower side lobes in the harmonic compared with the fundamental.

Harmonic imaging with 20 MHz transmit and 40 MHz receive showed capability superior to that of fundamental imaging at 20 MHz to diagnose retinal disease in the posterior segment of the eye. The center frequencies of transmit and receive elements of dual element transducers can be further optimized to match the designed center frequencies to support a larger dynamic range. The aperture size of transmit and receive elements can also be optimized with further experimentation to achieve the best combination of transmit and receive efficiency.

There is a need to form a high precision image of the prostate from outside the patient's body wherein the resolution is sufficient to image, for example, cancerous lesions on the surface of the prostate. To achieve such images, coded excitation, tissue harmonic imaging, advanced transducers operating in the 10 MHz to 20 MHz range are required to achieve a useable signal-to-noise reflection while being able to position the imaging transducer as close as possible to the prostate without risk or discomfort to the patient.

FIG. 8 is a schematic of the transducer face of a prior art multi-element ultrasound transducer configuration (taken from a slide show entitled “Ultrasound transducers by Ravi Managuli). This type of transducer can also be used in precision arc scanner of FIG. 6 discussed in the previous section.

As discussed in “High-Frequency Ultrasonic Imaging of the Anterior Segment Using an Annular Array Transducer” Ronald H. Silverman, Jeffrey A. Ketterling and D. Jackson Coleman, Ophthalmology. April 2007, very-high-frequency ultrasound (VHFU >35 MHz) allows imaging of anterior segment structures of the eye with a resolution of less than 40 microns. The low focal ratio of VHFU transducers, however, results in a depth-of-field of less than 1,000 microns (1,000 microns is equal to 1-mm). A dual element high-frequency annular array transducer for ocular imaging shows improved depth-of-field sensitivity and resolution compared to conventional single element transducers.

As also discussed in the preceding reference, a spherically curved multiple annular array ultrasound transducer was tested wherein the array consisted of five concentric rings of equal area, had an overall aperture of 6 mm and a geometric focus of 12 mm. The nominal center frequency of all array elements was 40 MHz. An experimental system was designed in which a single array element was pulsed and echo data recorded from all elements. By sequentially pulsing each element, echo data were acquired for all 25 transmit/receive annular combinations. The echo data were then synthetically focused and composite images produced. This technology offers improved depth-of-field, sensitivity and lateral resolution compared to single element fixed focus transducers and dual element annular array transducers currently used for VHFU imaging of the eye.

Factors that impact upon the overall utility of ultrasound systems include resolution, penetration, speed (frames/second), sensitivity (signal/noise) and depth-of-field. Resolution generally improves (and penetration declines) with frequency. Very-high-frequency (>35 MHz) ultrasound (VHFU) provides an axial resolution of <40 μm, allowing exquisitely detailed depiction of anatomic structures. However, attenuation at this frequency is high, even in water, limiting clinical imaging in this frequency range to the anterior segment.

Annular arrays can be fabricated with no curvature (i.e., flat) with a spherical lens, or with a spherical geometry. While the principle of dynamic focusing is the same for all, spherically curved devices are advantageous compared to flat arrays because fewer elements are required to achieve the same improvement in depth of field. Spherical curvature also leads to better lateral resolution for two transducers of similar aperture and number of elements.

Current VHFU systems for evaluation of the anterior segment of the eye are constrained by their very limited depth of field. This results in reduced sensitivity and degraded resolution outside a focal zone that measures under one millimeter in axial extent. The performance of an annular array transducer operating in the same frequency range as current single-element UBM systems showed that this technology can provide a six-fold increase in depth of field. The improved resolution and sensitivity offered by annular array technology can therefore provide significant practical advantages in diagnostic imaging of anatomy and pathology. Furthermore, this technology can be readily extended to lower frequencies, such as 20-25 MHz, that would allow improved assessment of pathologies. In summary, a 40-MHz multiple annular array transducer for imaging of the anterior and posterior segments can be fabricated to achieve improved depth-of-field, sensitivity and lateral resolution.

Spatial resolution in an ultrasonic imaging system is dependent on beam and focal properties of the source, tissue attenuation, non-linearity of the medium, tissue inhomogeneity, and speed of sound speed in each tissue region.

In ultrasound, axial resolution is improved as the bandwidth of the transducer is increased, which typically occurs for higher center frequencies. However, the attenuation of sound typically increases as frequency increases, which results in a decrease in penetration depth. Therefore, there is an inherent tradeoff between spatial resolution and penetration in ultrasonic imaging.

One way to increase the penetration depth without reducing axial resolution is by increasing the excitation pulse amplitude. However, increased excitation amplitude results in increased pressure levels that could result in unwanted heating or damage to tissues. Therefore, increasing the excitation pulse amplitude is not always a viable solution, depending on the region being imaged. For example, regulations for ultrasound power and time duration are low for the eye relative to the heart.

### Coded Excitation

Coded excitations are engineered excitation pulses that are capable of increasing the effective penetration depth of a transmitted signal in echo location imaging systems such as radar, sonar and ultrasound, by improving the signal-to-noise ratio (SNR).

An alternate solution that may be employed by the scanner of FIG. 6 is to increase the penetration depth, as opposed to increasing the excitation pulse amplitude, by increasing the excitation pulse duration by using coded excitation, thereby increasing the total transmitted energy and allowing for the substantial minimization of the transmitted peak power. However, increasing signal duration has the negative effect of decreasing the axial resolution of the ultrasonic imaging system.

To restore the axial resolution after excitation with a coded signal, pulse compression is used. Pulse compression can be realized by using one or more filtering methods. The main disadvantage of using coded excitation and pulse compression would be the introduction of range side lobes that can appear as false echoes in an image. The introduction of range side lobes is a detriment to ultrasonic image quality because it can reduce the contrast resolution. The main advantage for using coded excitation is that it is known to improve the echo signal-to-noise ratio by increasing the time/bandwidth product of the coded signal. This improvement in echo signal-to-noise results in greater depth of penetration in the range of a few centimeters for ultrasonic imaging and improved image quality. Furthermore, this increase in penetration depth allows the possibility of shifting to higher frequencies with larger bandwidths in order to increase the spatial resolution at depths where normally it would be difficult to image.

Ultrasound imaging is a non-ionizing, non-invasive, real-time imaging method when compared to other techniques such as magnetic resonance imaging. However, the finer resolution advantages offered by high frequency ultrasound are offset by limitations in penetration depth caused by frequency-dependent attenuation and limitations in depth-of-field when low f-number transducers are employed to improve cross-range resolution. Attenuation of ultrasound in tissue increases with frequency and, therefore, current uses of high frequency ultrasound are limited to applications that do not require deep penetration to image the tissue of interest. High frequency ultrasound image quality can be significantly improved by using two independent approaches.

The first approach uses synthetic focused annular arrays with overall apertures similar to typical spherically focused transducers to increase depth-of-field. The radial symmetry of annular arrays leads to a high-quality radiation pattern while employing fewer elements than linear or phased arrays. However, annular arrays need to be mechanically scanned to obtain a 2D image.

An annular array ultrasound transducer can consist of a two element array such as shown in FIG. 7B or a multi-element array such as shown in FIG. 8.

As an example, concentric annular type dual element transducers for second harmonic imaging at 20 MHz/40 MHz were designed to improve spatial resolution and depth of penetration for ophthalmic imaging applications. The outer ring element may be designed to transmit the 20 MHz signal and the inner circular element may be designed to receive the 40 MHz second harmonic signal. These types of annular arrays are described, for example, in “20 MHz/40 MHz Dual Element Transducers for High Frequency Harmonic Imaging, Kim, Cannata, Liu, Chang, Silverman and Shung, IEEE Transactions on Ultrasonics, Ferroelectrics and Frequency Control, \Vol. 55; NO. 12, December 2008.

A multi-annuli array transducer is described in “Chirp Coded Excitation Imaging with a High-frequency Ultrasound Annular Array”, Mamou, Ketterling and Silverman, IEEE Trans Ultrasonics, Ferroelectrics and Frequency Control. 2008 February 2008. The array consists of five equal-area annuli with a 10-mm total aperture and a 31-mm geometric focus.

The second high frequency ultrasound imaging approach uses coded excitations (i.e., engineered excitation pulses) that are capable of increasing the effective penetration depth by improving the signal-to-noise ratio. Resolution and penetration depth are critically important for medical ultrasound imaging. Normally, these two properties present a tradeoff, in which one property can be improved only at the expense of the other. However, it has been demonstrated that coded excitation is capable of extending the limit associated with this tradeoff. Coded excitation permits the signal-to-noise ratio to be increased through appropriate encoding on transmit and decoding on receive. In a published study, linear chirp signals were used to excite an annular array transducer. The objectives of this study were to demonstrate that chirp annular array imaging can lead to better image quality than current state-of-the-art high frequency ultrasound images. The described methods are general and are applicable to a vast range of clinical applications, including ophthalmological, dermatological, and gastrointestinal imaging.

To appreciate how coded excitation can increase signal-to-noise ratio (SNR), white noise can be added to the received response. Typically, a response had an SNR of 45 dB, which is in the range of most ultrasound imaging systems. Chirp excitations led to an increase in SNR of greater than 14 dB.

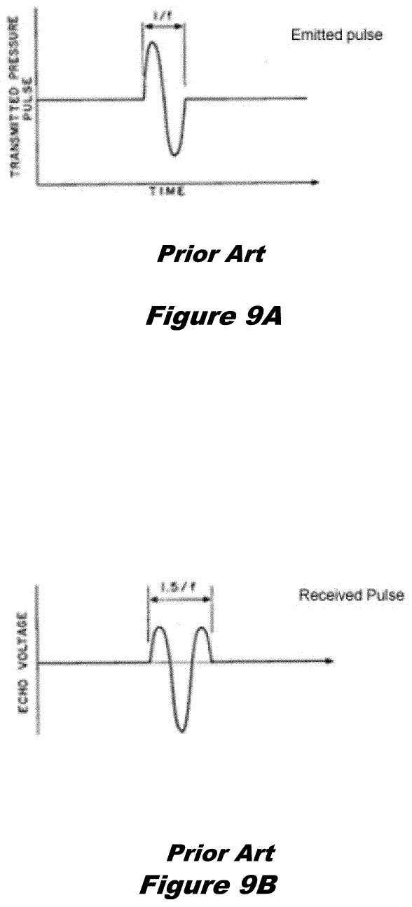

FIGS. 9A and 9B are examples of a simple emitted and received ultrasound pulse waveform, respectively, that are used, for example, in the precision arc scanner described above with reference to FIG. 6 which uses a single element transducer such as shown in FIG. 7A which is taken from “Ultrasonography of the Eye and Orbit”, Second Edition, Coleman et al, published by Lippincott Williams & Wilkins, 2006.

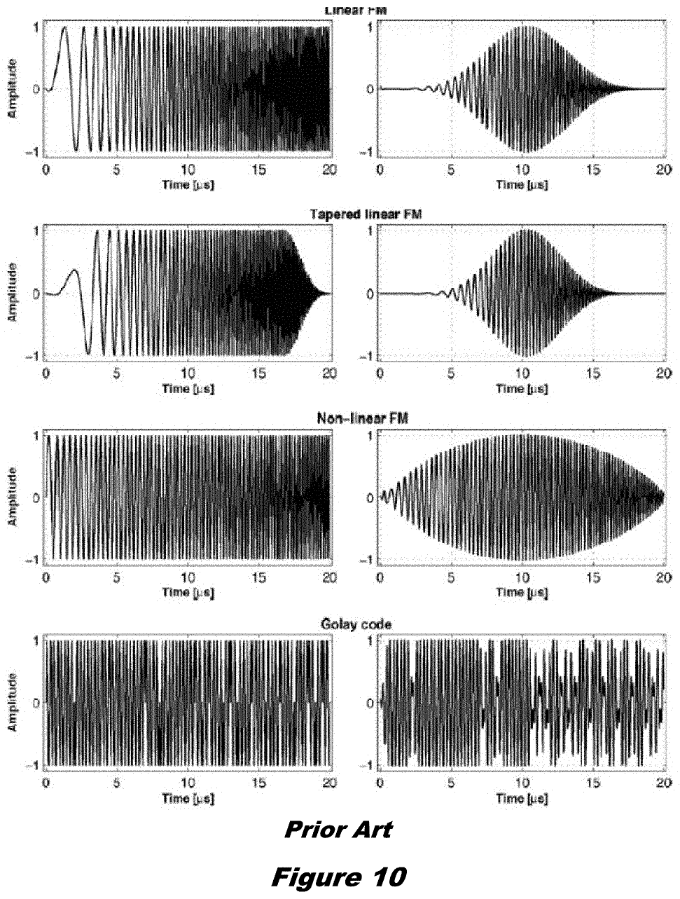

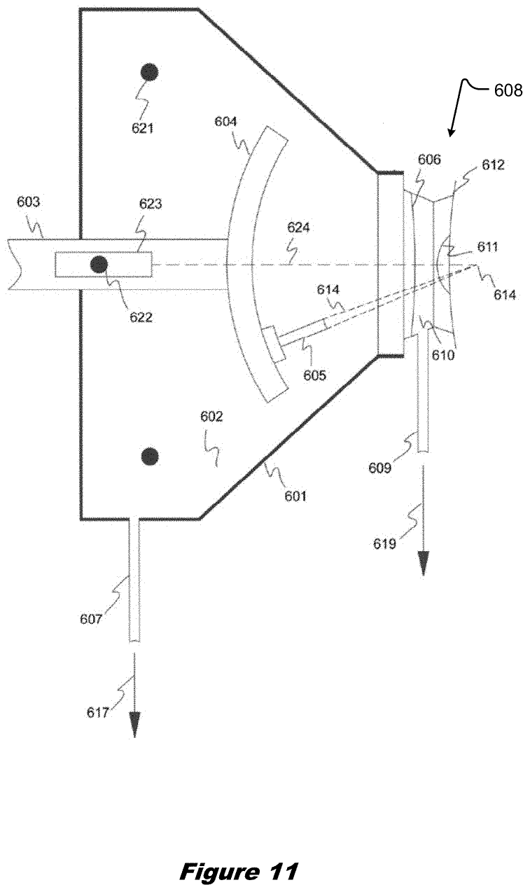

FIG. 10 shows examples of several emitted coded excitation ultrasound pulse waveforms. This figure was taken from “Use of Modulated Excitation Signals in Medical Ultrasound. Part I: Basic Concepts and Expected Benefits”, Misaridis and Jensen, IEEE Transactions on Ultrasonics, Ferroelectrics, and Frequency Control, vol. 52, no. 2, February 2005.

FIG. 11 is a schematic illustration of an arc scanning device of another embodiment of the disclosure. This figure illustrates positioning of an imaging and/or therapeutic irradiating transducer along an arc guide whose center of curvature is centered approximately on the center of curvature of an eye component of interest. FIG. 11 shows fixation lights 621 and 622 that allow the patient to fixate his or her eye to maintain it in a steady position during scanning. FIG. 11 also shows an optical video camera 623 which may be used by the operator of the arc scanner to monitor the position of the patient's eye and to determine whether the patient's eye is open before a scan is initiated. When the patient fixes and eye on light 622, the visual axis of the eye 624 will align with the optical video camera 623.

The imaging and/or therapeutic irradiating transducer and its arc guide assembly are positioned in a chamber 601 and are immersed in a medium suitable for conducting acoustic energy in the form of ultrasound such as water 602 to provide a transmission path for the acoustic signals. The patient's eye must also be immersed in water to provide continuity of the transmission path for the acoustic signal. This is accomplished by using a detachable eyepiece 608. FIG. 11 also shows a hygienic barrier 606 which separates the water chamber 601 in which the transducer 605 and arc scanning positioner, scan head and transducer carriage assembly 604 are immersed from the water 610 in which the patient's eye is immersed. This barrier 606 separates the water chamber 601 in which the transducer 605 and transducer carriage assembly 604 are contained from the water 610 in which the patient's eye is immersed. The arc guide assembly and associated components may be contaminated, for example, by particles from wearing mechanical components. The water 610 in which the patient's eye is immersed may be contaminated by bacteria or virus particles from the patient. As can be appreciated, the water 610 in which the patient's eye is immersed should be changed for every patient to prevent possible disease transmission. As can be further appreciated, the hygienic barrier 606 must be substantially transparent to ultrasound so as to maintain a clear acoustic transmission path between the patient's eye and the ultrasonic transducer 605. The hygienic barrier 606 is typically formed as part of a disposable eyepiece.

References are made herein to a medium suitable for conducting acoustic energy in the form of ultrasound. There are reasons to prefer that the medium be pure water or physiologic saline (also known as normal saline) but the embodiments do not exclude other media suitable for conducting acoustic energy in the form of ultrasound. Most other media present an increased danger to the patient's eye, even with a barrier interposed between the eye and the ultrasonic transducer. Barriers can leak or be breached, allowing the liquids on either side to mix, thus bringing a potentially harmful material into contact with a patient's eye.

It should be appreciated, however, that non-harmful, less-corrosive media and leak-proof, impenetrable barriers might be developed or discovered. This might allow different media other than pure water or physiologic saline to be used in this disclosure. Nothing about embodiments herein other than the hazards just described requires pure water or physiologic saline to be present in the chamber containing the transducer. All references herein to water should accordingly be understood as referring to any suitable liquid.

FIG. 11 further illustrates the continuity of an acoustic transmission path through water. A positioning arm 603 and transducer carriage assembly 604 on which the ultrasonic transducer 605 is mounted are positioned in the chamber 601 of water 602. An ultrasonically transparent hygienic barrier 606 separates chamber 601 from the interior of an eyepiece 608. The eyepiece 608 contains a separate volume of water 610 (typically a saline solution which is preferably sterilized) which fills the interior of the eyepiece 608 and contacts a patient's eye surface 611. The eyepiece 608 is connected and sealed to the main chamber 601 of the arc scanning device, and is also sealed against the patient's face 612. As can be seen, there is a continuous path through water from the transducer 605 to the patient's eye surface 611 for the efficient passage of acoustic energy 614. The hygienic barrier 606 readily passes acoustic energy without alteration, thus forming a portion of the continuous path between the transducer 605 and the patient's eye surface 611. Since the acoustic impedance of the patient's eye is approximately that of water, the acoustic energy from the transducer can be efficiently transmitted into the eye and reflected back from an eye component, such as for example, the surface of the cornea, to the transducer. Also shown in FIG. 11 are a water fill tube 607 for the main chamber 601 and a separate water fill tube 609 for the eyepiece 608. Fill tubes 607, 609 may be used to add or remove water from the chamber and eyepiece as indicated by arrows 617 and 619. As can be appreciated, the water used in the eyepiece can be distilled or slightly saline to match the salinity of the eye, and the water used in the eyepiece can be introduced at a temperature that is comfortable for the patient.

As can be appreciated, the fluid in the eyepiece 608, the hygienic membrane or barrier 606 and the water in the main chamber 601 are preferably optically transparent to allow the video camera 623 to image the patient's eye and to allow the fixation light sources 621, 622 to be seen by the patient being scanned.

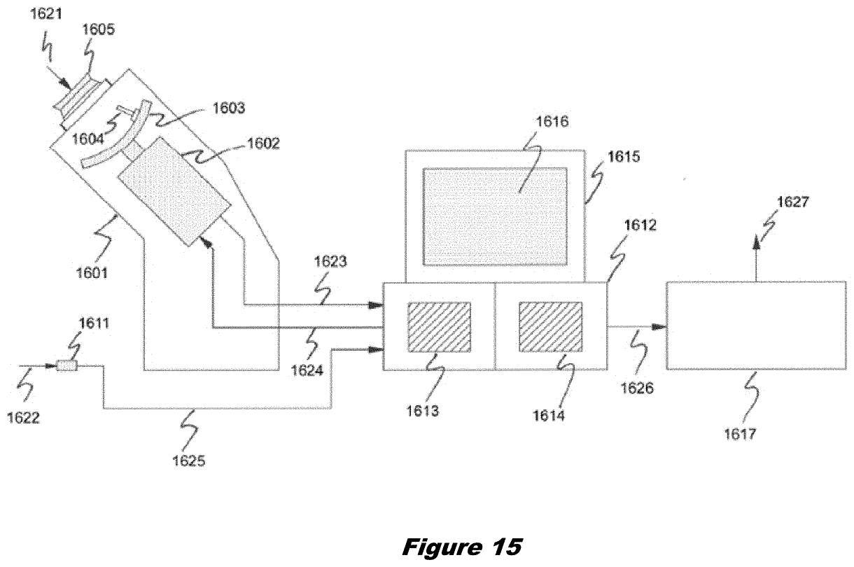

The arc scanning device includes a control and signal processing system which is not illustrated in FIG. 11. The control and signal processing system is described in FIG. 15, below.

### Tissue Harmonic Imaging

Tissue harmonic imaging exploits non-linear propagation of ultrasound through body tissues. The high pressure portion of the wave travels faster than low pressure resulting in distortion of the shape of the wave. This change in waveform leads to generation of harmonics (multiples of the fundamental or transmitted frequency) from the tissue. Typically, the second harmonic is used to produce the image as the subsequent harmonics are of decreasing amplitude and hence insufficient to generate a proper image. These harmonic waves that are generated within the tissue increase with depth to a point of maximum intensity and then decrease with further depth due to attenuation. Hence the maximum intensity is achieved at an optimum depth below the surface. Advantages over conventional ultrasound include: decreased reverberation and side lobe artifacts; increased axial and lateral resolution; increased signal-to-noise ratio; and improved resolution in patients with large body habitus.

Tissue harmonic ultrasound imaging has been accepted as one of the standard imaging modalities in many applications since its introduction to medical ultrasound imaging in the 1990s. Especially in cardiac and abdominal studies, tissue harmonic imaging is very often used for diagnostics along with fundamental imaging. By utilizing the second harmonic component of the received signal, images can be improved by reducing near field reverberation, decreasing phase aberration error, and improving border delineation.

Ultrasound tissue harmonic imaging utilizing nonlinear distortion of the transmitted frequencies within the body is useful for producing a sharper, higher-contrast ultrasound image than that of the fundamental frequency. Due to its improved conspicuity (the property of being clearly discernible) and border definition, tissue harmonic imaging has been widely used for detecting subtle lesions in, for example, the thyroid and breast, and visualizing technically-challenging patients with high body mass index. However, compared to conventional ultrasound imaging, tissue harmonic imaging suffers from the low signal-to-noise ratio, resulting in limited penetration depth. The signal-to-noise ratio in tissue harmonic imaging can be substantially increased by utilizing coded excitation techniques, such as described previously in this disclosure. In coded tissue harmonic imaging, similar to conventional coded excitation, specially-encoded ultrasound signals (for example, Barker, Golay and chirp) are transmitted, and then back-scattered receive signals containing fundamental and harmonic frequencies are selectively decoded via pulse compression.

### Tissue Harmonic Imaging and Coded Excitation Together

Tissue harmonic imaging allows one to obtain medical ultrasound images with higher signal-to-noise ratio and higher spatial resolution. Tissue harmonic imaging and coded excitation together have been applied to medical ultrasound imaging. Coded excitation can overcome the trade-off between spatial resolution and penetration, which occurs when using a conventional transmitted pulse. For example, a chirp signal is frequently used for medical ultrasound imaging. A combination of coded excitation and tissue harmonic imaging has been found to produce superior ultrasound images.

As discussed in “Use of Modulated Excitation Signals in Medical Ultrasound. Part I: Basic Concepts and Expected Benefits”, Misaridis and Jensen, IEEE Transactions on Ultrasonics, Ferroelectrics, and Frequency Control, vol. 52, no. 2, February 2005, tissue harmonic imaging allows one to obtain medical ultrasound images with higher signal-to-noise ratio and higher spatial resolution. Tissue harmonic imaging and coded excitation applied to medical ultrasound imaging has been investigated. Coded excitation can overcome the trade-off between spatial resolution and penetration, which occurs when using a conventional transmitted pulse. For example, a chirp signal is frequently used for medical ultrasound imaging. A combination of coded excitation and tissue harmonic imaging has been found to produce superior ultrasound images.

As discussed in “Coded Excitation for Ultrasound Tissue Harmonic Imaging”, Song, Kim, Sohn, Song and Yoo. Received in revised form 18 Dec. 2009 Ultrasonics journal homepage: www.elsevier.com/locate/ultras, it is shown how coded signals, when processed with a matched filter, can be evaluated in the presence of ultrasonic attenuation using ambiguity functions. It is shown that if matched-filter receiver processing is used, the compressed output is not the autocorrelation function of the code, but a cross section of the ambiguity function for a certain frequency downshift. Therefore, the AF of the transmitted waveform ought to have desired properties in the entire delay-frequency shift plane. The criteria of selecting the appropriate coded waveforms and receiver processing filters have been discussed in detail. One of the main results is the conclusion that linear FM signals have the best and most robust features for ultrasound imaging. Other coded signals such as nonlinear FM and binary complementary Golay codes also have been considered and characterized in terms of SNR and sensitivity to frequency shifts. These results have been demonstrated. It is found that, in the case of linear FM signals, a SNR improvement of 12 to 18 dB can be expected for large imaging depths of attenuating media, without any depth dependent filter compensation. In contrast, nonlinear FM modulation and binary codes are shown to give a SNR improvement of only 4 to 9 dB when processed with a matched filter. It was shown how the higher demands on the codes in medical ultrasound can be met by amplitude tapering of the emitted signal and by using a mismatched filter during receive processing to keep temporal side lobes below 60 to 100 dB.

### Imaging and Irradiating Transducers

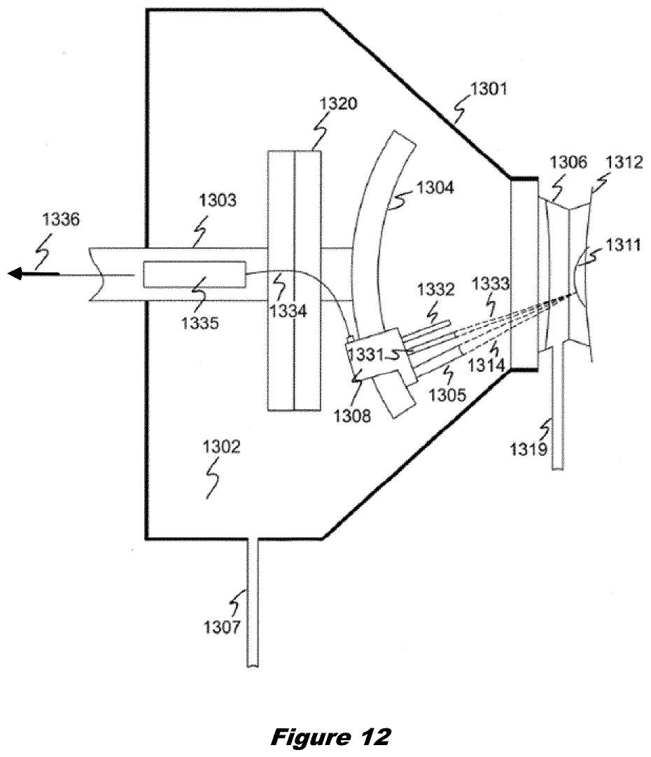

FIG. 12 illustrates an embodiment of a combined imaging transducer and higher powered ultrasound irradiating transducer, both mounted on the same carriage as part of a scan head. The scan head is positioned with respect to a patient's eye using a positioner mechanism. The scan head may include a probe carriage for moving the imaging and irradiating probes. The positioner mechanism, the scan head, and probe carriage may be immersed in water. A disposable eyepiece may be connected to the system and filled separately with water to provide a continuous water transmission path from the probes to the surface of patient's eye. As discussed above, all references to water in all embodiments shall be understood to refer to any medium suitable for conducting acoustic and optical energy.

The general components of an embodiment of a combined imaging transducer and higher powered ultrasound irradiating transducer are shown in FIG. 12 with particular attention to the components in the scanning head which are unique to the combined imaging system.

A chamber 1301 of water 1302 is shown with a positioning arm 1303, a linear guide track 1320, and an arcuate guide assembly 1304 on which a probe carriage 1308 is mounted. The positioning arm 1303 may rotate about a longitudinal axis 1336 which passes generally through a center of the positioning arm 1303 and which is substantially perpendicular a rear wall of the chamber 1301. The positioning arm 1303 may also move back and forth axially in the direction of the longitudinal axis 1336. The linear guide track 1320 is interconnected to the positioning arm 1303 and substantially perpendicular to the longitudinal axis 1336. The arcuate guide assembly 1304 is interconnected to the linear guide track 1320 and the arcuate guide assembly 1304 is substantially perpendicular to the longitudinal axis 1336. The arcuate guide assembly 1304 may move back and forth on the linear guide track 1320. The probe carriage 1308 is mounted on the arcuate guide assembly 1304 and may move in an arcuate motion along the arcuate guide assembly 1304. The motions of the positioning arm 1303, the linear guide track 1320, and the arcuate guide assembly 1304 can be controlled independently. Because of its connection to the positioning arm 1303, the linear guide track 1320, and the arcuate guide assembly 1304, the probe carriage 1308 may be rotated about the longitudinal axis 1336, may be move axially along the longitudinal axis 1336, and may be moved in a combination of linear and arcuate motions along the linear guide track 1320 and the arcuate guide assembly 1304.

An ultrasonically and optically transparent barrier (not shown) separates chamber 1301 from the interior of an eyepiece 1306. The eyepiece 1306 contains a separate volume of water which fills the interior of the eyepiece 1306 and contacts a patient's eye surface 1311. The eyepiece 1306 is connected and sealed to the main chamber 1301 of the scanning device, and is also sealed against the patient's face 1312.

By being both ultrasonically and optically transparent, the membrane can pass ultrasound energy from the ultrasound transducers and optical energy from the camera that monitors eye movement and eyelid position.

Also shown in FIG. 12 are a water fill tube 1307 for the main chamber 1301 and a separate water fill tube 1319 for the eyepiece 1306. As can be appreciated, the water used in the eyepiece can be distilled or slightly saline to match the salinity of the eye, and the water used in the eyepiece can be introduced at a temperature that is comfortable for the patient.

Probe carriage 1308 comprises an imaging ultrasound transducer probe 1305, an irradiating ultrasound transducer 1331. A third transducer holder 1332 is also shown but is usually not needed. The ultrasound imaging transducer probe 1305 and ultrasound irradiating probe 1331 are preferably focused at the same point on or within the patient's eye. Alternately, the probes 1305, 1331 can be substantially parallel and then offset by a small linear dimension. The ultrasound imaging and irradiating transducers 1305 and 1331 may be connected via a ultrasound cables (not shown) to the ultrasound recording apparatus (not shown).

FIG. 12 illustrates continuous acoustic 1314 and optical 1333 transmission paths for the efficient passage of acoustic and optical energy. The continuous paths proceeds from the probes 1305 and 1331 through water to the patient's eye surface 1311. The barrier separating the chamber 1301 and eyepiece 1306 readily passes acoustic and optical energy with minimal alteration, thus forming a portion of the continuous paths between the probes 1305 and 1331 and the patient's eye surface 1311. Since the acoustic and optical impedances of the patient's eye are approximately those of water, the energy from the probes can be efficiently transmitted into the eye and reflected back from an eye component, such as for example, the surface of the cornea, to the probe.

In a first configuration, the imaging transducer and irradiating therapeutic transducer are mounted on a revolver type holder. The imaging transducer may be rotated about a rotational axis into position with respect to the eye and, after precisely positioning the focal point of the imaging transducer on a target ocular feature, an image made of the eye. After confirming that the focal point of the imaging transducer is positioned properly on the target ocular feature, the irradiating transducer may be rotated into position about the same rotational axis with respect to the eye. The target ocular feature is then therapeutically irradiated. When the imaging and irradiating transducers have different focal points, the holder may require repositioning between the imaging and irradiating steps to ensure the focal point of the irradiating transducer is centered on the target ocular feature. Then the imaging transducer may be rotated back into position about the same rotational axis and an image made of the irradiated eye to determine if future therapeutic treatment is desired. This is typically done by comparing dimensions of selected ocular features before irradiation with dimensions of the selected ocular features after irradiation.

The irradiating transducer required for this task is typically in the range of about 17 mm diameter to about 30 mm in diameter, typically in the frequency range of about 5 MHz to about 20 MHz and typically has a focal length in the range of about 20 mm to about 40 mm. The imaging transducer is typically in the range of about 4 mm diameter to about 7 mm in diameter, typically in the frequency range of about 25MHz to about 40 MHz and typically has a focal length in the range of about 20 mm to about 40 mm. The irradiating transducer would likely require its own pulser/receiver board while the imaging transducer, which requires much less power, would have a separate pulser/receiver board.

In a second configuration, a common transducer in a first mode acts as the imaging transducer and in a second mode acts as the irradiating therapeutic transducer. The transducer may be mounted in a conventional holder. The irradiating transducer required is typically in the range of about 17 mm diameter to about 30 mm in diameter, typically in the frequency range of about 5 MHz to about 20 MHz and typically has a focal length in the range of about 20 mm to about 40 mm.

When operating in this second configuration, coded excitation and tissue harmonic imaging techniques may be used to image the irradiated tissue. For example, a 15 MHz irradiating transducer would produce a strong second harmonic at about 30 MHz that could be used for imaging. As noted previously, tissue harmonic ultrasound imaging has been used in medical ultrasound imaging since the 1990s.

As can be appreciated, the first configuration can be operated to include coded excitation and tissue harmonic imaging techniques to produce images at different frequencies and/or with different focal length transducers. For example, an imaging transducer and an irradiating therapeutic transducer can be mounted in a revolver type holder. The irradiating transducer could be about a 12 MHz transducer with a focal length of about 20 mm to about 40 mm that would produce a strong second harmonic at about 24 MHz that could be used for imaging. The imaging transducer with a focal length of about 10 mm to about 20 mm typically operates in the range of about 2 5MHz to about 40 MHz.

Forms of coded excitation, such as described above, can enhance ultrasound imaging by improving the measurement resolution of the radii of curvature of the anterior and posterior cornea and lens and the thicknesses and separations of the cornea and lens. While not wishing to be bound by any theory, it is believed that forms of coded excitation can enhance ablation of the ciliary body and the vibration of the trabecular mesh.

This, in turn, can reduce the production of the fluid by the ciliary body that is responsible for the pressure in the eye and improve the flow through the trabecular mesh of the fluid produced by the ciliary body.

In one embodiment, the present disclosure describes a method treatment of a human eye for elevated intraocular pressure comprising imaging the anterior segment of an eye over a first range of ultrasound frequencies and amplitudes to confirm positioning of the focal point of the transducer on the ciliary body and yield selected ocular component measurements; then ablating the ciliary body over a different second range of ultrasound frequencies and amplitudes; vibrating the trabecular mesh over a different third range of ultrasound frequencies and amplitudes; and re-imaging the anterior segment of an eye over the first range of ultrasound frequencies and amplitudes to determine any dimensional changes in the components of the eye resulting from ablation of the ciliary or vibration of the trabecular mesh and the parameters to be used in a further therapeutic treatment step (e.g., time and frequency of ultrasound emissions). The changes in the dimensional changes are related to the intraocular pressure and indicate whether further therapeutic treatment is necessary.

In one embodiment, after imaging and ultrasound ablation/trabecular mesh vibration, a tonometer (such as using one or more of the tonometry techniques listed above) is positioned over a portion of the cornea after each treatment stage to measure directly IOP. The correct positioning of the tonometer by the computer can be done using the images generated by the ultrasound imaging step(s) in which a cornea surface is detected and mapped. The tonometer can be positioned on an opposing side of a fluid separation membrane in the eye piece from the arcuate guide assembly 1304. The measured IOP is compared against a target IOP or IOP range to determine whether further therapeutic treatment is necessary and if so the parameters to be used in a further therapeutic treatment step (e.g., time and frequency of ultrasound emissions).

One embodiment is based on the known relationships between pupil/iris ratio, and sclera contour features with IOP. Specifically, it is known that the pupil/iris diameter ratio is directly proportional to the IOP values in mmHg while the sclera contour features (contour height, distance, contour area, and angle) are inversely proportional to IOP. The angle between the back of the sclera and/or cornea and the front of the iris and the trabecular-iris angle or TIA are also proportional to the IOP. In this embodiment, before and after imaging and ultrasound ablation/trabecular mesh vibration, ultrasound imaging determines the pupil/iris ration and/or a common sclera feature to measure indirectly IOP. The measured IOP is compared against a target IOP or IOP range to determine whether further therapeutic treatment is necessary and if so the parameters to be used in a further therapeutic treatment step (e.g., time and frequency of ultrasound emissions).

One embodiment is based on a coefficient of reflection ultrasound energy from a targeted ocular structure. The technique directs ultrasound energy at the target ocular structure from a predefined incident angle (relative to a measured axis of the eye) and measures the coefficient of reflection. As the internal pressure of an object increases, the reflection coefficient increases. In this embodiment, before and after imaging and ultrasound ablation/trabecular mesh vibration, ultrasound imaging determines the coefficients of reflection to measure indirectly IOP. The measured IOP is compared against a target IOP or IOP range to determine whether further therapeutic treatment is necessary and if so the parameters to be used in a further therapeutic treatment step (e.g., time and frequency of ultrasound emissions).

FIG. 5B is a schematic of some of the typical dimensions of the human eye in millimeters and these dimensions apply at least along or near the optical axis.
Thickness of cornea˜0.5 mm
Radius of curvature anterior cornea surface˜7.7 mm
Radius of curvature posterior cornea surface˜6.8 mm
Distance from the front of the cornea to the front of the lens˜3.3 mm
Thickness of lens˜3.5 mm
Radius of curvature anterior lens surface˜11 mm
Radius of curvature posterior lens surface˜-6.0 mm
Equatorial diameter of lens˜8.5 mm to 10 mm
Distance from the rear of the lens to the front of the retina˜16 mm

These are representative dimensions of the relaxed eye. The distance from the front of the cornea to the front of the lens along the optical axis and the thickness of lens along the optical axis depend upon accommodation. These values were taken from “Optics of the Human Eye”, D. A. Atchison, G. Smith, Robert Stevenson House, Edinburgh, ISBN 0 7506 3775 7, first printed in 2000.

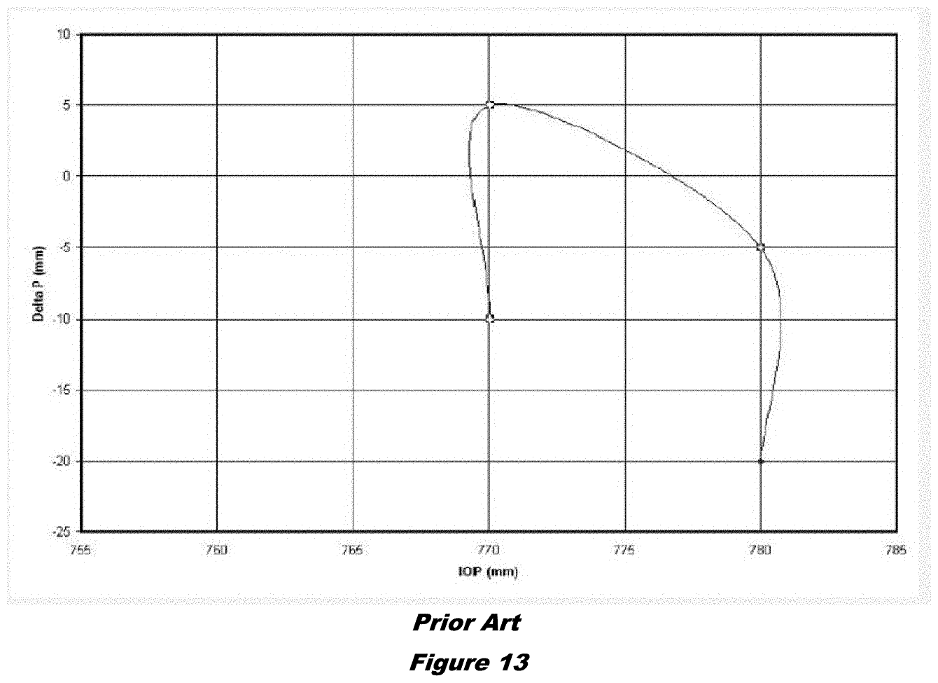

Possible sequences of operations to lower IOP using an imaging transducer and higher powered ultrasound irradiating transducer such as illustrated in FIG. 13 include the following:

In a first set of operations, the eye to be treated is imaged with the imaging transducer over a first range of ultrasound frequencies and amplitudes to detect and determine a parameter of a target ocular component (e.g., a dimension, shape, area, and/or volume of the ciliary body, the radii of curvature of the anterior and posterior cornea and lens; the cornea and lens thicknesses; the on-axis distances between the anterior surface of the cornea to the anterior surface of the lens and/or between the posterior surface of the lens to the anterior surface of the retina, the pupil/iris ratio, and/or a sclera contour feature).

The ciliary body is ablated with the irradiating transducer over a second range of ultrasound frequencies and amplitudes.

Optionally the target ocular component of the eye to be treated is again imaged with the imaging transducer over the first range of ultrasound frequencies and amplitudes to detect and determine the parameter of the target ocular component. Any parameter changes are determined.

Vibrate the trabecular mesh over a third range of ultrasound frequencies and amplitudes.

The eye to be treated is imaged with the imaging transducer over the first range of ultrasound frequencies and amplitudes and note the parameter of the target ocular component. Any parameter changes are determined.

The parameter changes can be correlated to the anterior surface of the retina with pressure deferential across the cornea and reduction in IOP.

The steps of ablating, imaging, vibrating and imaging can be repeated as necessary to produce the target IOP reduction and/or final IOP.

The above treatment protocol can be used together with any other method of determining IOP. Alternatively or additionally, one or more the imaging steps could be replaced with a tonometry technique to determine IOP and/or coefficient of reflection analysis.

This method can be implemented using a dual element or an annular array transducer or it can be implemented by separate single element transducers on a revolver type transducer mount.

### Intra Ocular Pressure (IOP)

Normal intraocular eye pressure ranges from about 12 to about 22 mm Hg. An intraocular eye pressure of greater than about 22 mm Hg is considered higher than normal. An average value of intraocular pressure is 15.5 mm Hg with fluctuations of about 2.75 mm Hg.

The external pressure on an eye can be increased above atmospheric pressure by raising or lowering the saline bag by an inch or two above the patient's eye. This pressure change can change the shape of the cornea or globe or the sclera. Pressure on the outside of the eye can be changed by about 1 mm Hg for every half inch of saline bag elevation. (average IOP is ˜15.5 mm Hg).

This combination cited in the above example could be used to irradiate the target eye with non ionizing ultrasound while taking images of the target eye at 12 MHz, 24 MHz, and 40 MHz.

FIG. 13 is a graph of intraocular pressure (IOP) of a human eye expressed as millimeters of mercury versus water pressure expressed as inches of water column height. A millimeter of mercury (Hg) is taken as 12*33.95/760 or 407 inches of water (H₂O). This graph is applicable to estimating the external pressure on an eye when using an ultrasound eye piece or ultrasound imaging goggles when the eye piece or goggles are filled with saline solution from a saline bag positioned above the head of the patient.

I=M*I*f/m

where: I=pressure in inches of water
M=pressure in millimeters of mercury
i=inches per foot
f=feet of water per atmosphere of pressure
m=millimeters of mercury per atmosphere of pressure

The external pressure on an eye when using an ultrasound eye piece or ultrasound imaging goggles may also be applied from a saline reservoir that is pressurized to a specified pressure between about 12 to about 22 mm Hg. As external pressure is being applied to the outside of the eye, the eye can be simultaneously imaged by ultrasound to determine any resulting change in radius of curvature of the cornea or lens or globe of the eye.

Pₒᵤₜ=external pressure
Pᵢₙ=internal eye pressure (IOP)
delta p=Pₒᵤₜ−Pᵢₙ
in natural state, Pont is less than Pin and the difference is delta punt

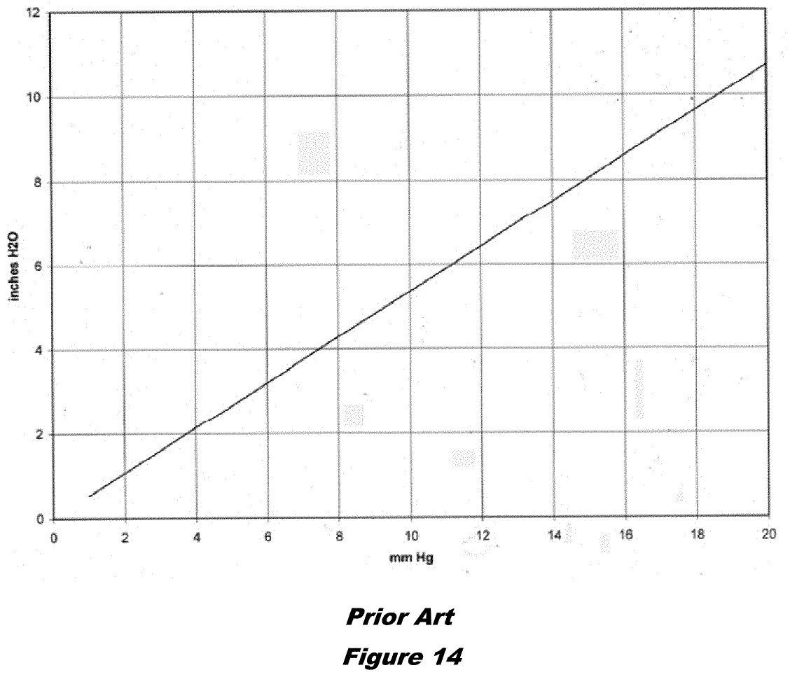

FIG. 14 is a graph of pressure differential across the cornea versus IOP The pressure differential is the pressure difference between the fluid on the anterior of the cornea and the IOP. This is usually negative as IOP is usually higher than ambient pressure. If ambient pressure is taken to be 760 mm Hg, then normal intraocular eye pressure ranges from about 772 to about 782 mm Hg. An intraocular eye pressure of greater than about 782 mm Hg is considered higher than normal. An average value of intraocular pressure is 775.5 mm Hg with fluctuations of about 2.75 mm Hg.

### High-Intensity Focused Ultrasound

High-intensity focused ultrasound (HIFU) is a non-invasive therapeutic technique that uses non-ionizing ultrasonic waves to heat tissue. HIFU can be used to increase the flow of blood or to destroy tissue, such as tumors, through a number of mechanisms. The technology is similar to ultrasonic imaging, although practiced at lower frequencies and higher acoustic power. Acoustic lenses may be used to achieve the necessary intensity at the target tissue without damaging the surrounding tissue. “Systematic Review of the Efficacy and Safety of High-Intensity Focused Ultrasound for the Primary and Salvage Treatment of Prostate Cancer”, M. Warmuth, T. Johansson, P. Mad, European Urology 58 (2010) 803-815, Sep. 17, 2010.

A typical HIFU transducer has a diameter of about 19 mm with a center frequency of about 5 MHz, a focal length of about 15 mm and a focal intensity of about 200 W/mm². Another typical HIFU transducer has a diameter of about 60 mm with a center frequency of about 1 MHz, a focal length of about 75 mm and a focal intensity of about 17 W/mm².

### Control and Signal Processing

FIG. 15 is a schematic representation of an arc scanning system according to an embodiment of the disclosure. The arc scanning apparatus is comprised of an arc scanning machine 1601 which includes an arc guide positioning mechanism 1602, an arc guide (or arcuate guide or arc track) 1603, an ultrasonic transducer 1604 and a disposable eyepiece 1605. The arc scanning apparatus may also include a more complex scan head in which an arcuate guide track is mounted on a linear guide track.

The arc scanning machine 1601 is connected to a computer 1612 which includes a processor module 1613, a memory module 1614 and a video monitor 1615 with video screen 1616. The computer 1612 is connected to an operator input device such as a mouse 1611, and/or a keyboard (not shown) or speech recognition device. The computer 1612 is also connected to an output device such as, for example, a printer or internet connection 1617. The patient is seated at the machine 1601 with one of their eyes engaged with disposable eyepiece 1605. The patient's eye component to be imaged is represented by input 1621. The operator using mouse and/or keyboard 1611 and video screen 616 or speech recognition device, inputs information into computer 1612 selecting the type of scan and scan configurations as well as the desired type of output image and analyses. The operator, using mouse and/or keyboard 1611 or speech recognition device, a video camera in scanning machine 1601 and video screen 1616, then centers a set of cross hairs displayed on video screen 1616 on the desired component of the patient's eye, also displayed on video screen 1616, setting one of the cross hairs as the prime meridian for scanning. Once this is accomplished, the operator instructs computer 1612 using either mouse and/or keyboard 1611 or speech recognition device to proceed with the scanning sequence. Now the computer processor 1613 takes over the procedure and issues instructions via path 1624 to the positioning head 1602, the arcuate track 1603 and the transducer carriage 1605 and receives positional and imaging data via path 1623 which is stored in memory module 1614. The computer processor 1613 proceeds with a sequence of operations such as for example: (1) rough focus transducer 1604 on the selected eye component; (2) accurately center arcuate track 1604 with respect to the selected eye component; (3) accurately focus transducer 1604 on the selected feature of the selected eye component; (4) rotate the arcuate track through a substantial angle and repeat steps (1) through (3) on a second meridian; (5) rotate the arcuate track back to the prime meridian; (6) initiate a set of A-scans along each of the of selected scan meridians, storing this information in memory module 1614; (7) utilizing processor 1613, converting the A-scans for each meridian into a set of B-scans and then processing the B-scans to form an image associated with each meridian; (8) performing the selected analyses on the A-scans, B-scans and images associated with each or all of the meridians scanned; and (9) outputting the data 1627 in a preselected format to an output device such as printer 1617. The output can also be stored in memory module 1614 for later retrieval on video screen 1616, or for transmission to remote computers or other output devices via any number of well-known data transmission means.

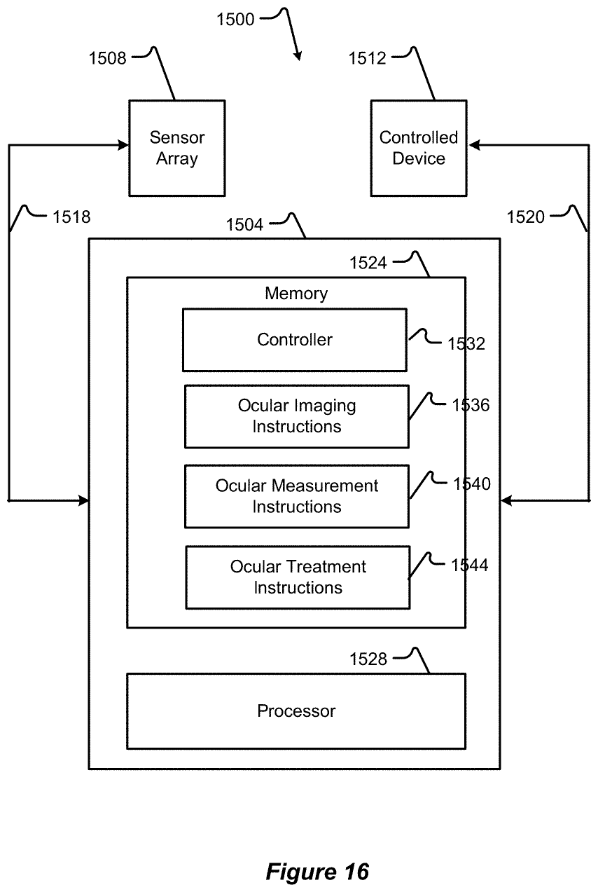

FIG. 16 depicts a control and signal processing system for any of the embodiments of the present disclosure discussed above. The system 1500 includes a sensor array 1508 and a controlled device 1512 in signal communication via, duplexed channels 1518 and 1520, with a computer 1504.

The sensor array 1508 comprises linear or angular position sensors that, among other things, track the relative and/or absolute positions of the various movable components and the alignment of various stationary and moveable components, such as, but not limited to, the one or more position tracking sensors, the positioning arms and probe carriage assembly, the fixation lights, the optical video camera, the arcuate guide assembly, the transducer probes, the probe carriage, the linear guide track, the motors to move the position arms, motors to move the arcuate guide assembly, and motors to move the probe carriage. The sensor array may comprise any suitable type of positional sensors, including inductive non-contact position sensors, string potentiometers, linear variable differential transformers, potentiometers, capacitive transducers, eddy-current sensors, Hall effect sensors, proximity sensors (optical), grating sensors, optical encoders (rotary or linear), and photo diode arrays. Candidate sensor types are discussed in U.S. Pat. No. 8,758,252.

The controlled device 1512 is any device having an operation or feature controlled by the computer 1504. Controlled devices include the various movable or activatable components, such as, but not limited to, the one or more position tracking sensors, the positioning arms, the transducer carriage assembly, the fixation lights, the optical video camera, the arcuate guide assembly, the imaging and irradiation transducer probe(s), the probe carriage, the linear guide track, the motors to move the position arms, motors to move the arcuate guide assembly, and motors to move the probe carriage.

The computer 1504 may comprise a software-controlled device that includes, in memory 1524, a number of sets of instructions executable by a processor 1528. The executable instructions include a controller 1532 to receive and process positioning signals from the sensor array 1508 and generate and transmit appropriate commands to the monitored controlled device 1512, ocular imaging instructions 1536 to receive and process A- and B-scan images to produce two-, three-, or four-dimensional images of selected ocular components or features, ocular measurement instructions 1540 to determine, as discussed above, the dimensions and positional relationships of selected ocular components and/or features that can indicate an irradiation treatment efficacy and/or IOP level, and ocular treatment instructions 1544 to therapeutically irradiate the target ocular component.

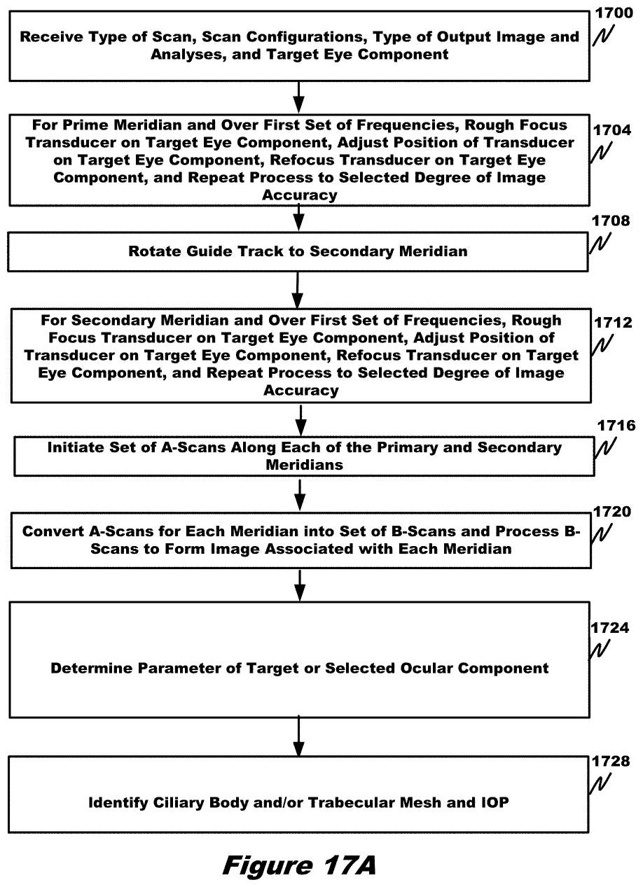

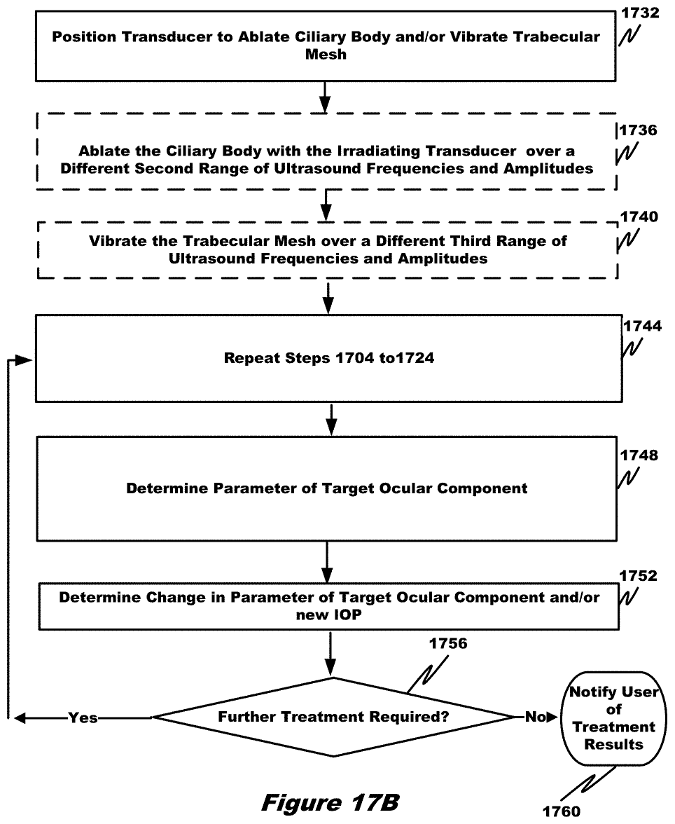

An embodiment of operations performed by the various instruction sets will be discussed with reference to FIGS. 17A-B. The instructions depict a possible sequence of operations to lower IOP using an imaging transducer and higher powered ultrasound irradiating transducer such as using the imaging systems of FIGS. 12-13.

With reference to FIG. 17A, the processor 1528 begins in step 1700 by receiving input from a clinician. The input comprises a type of scan to be performed, a scan configuration, a type of output image and analysis to be performed, and the target eye or ocular component to be treated. For example, the target ocular component can be a ciliary body or trabecular meshwork.

The processor 1528 next executes the ocular imaging instructions 1536 and causes the imaging transducer to image the target components of the eye to be treated over a first range of ultrasound frequencies and amplitudes. In one embodiment, the imaging operations include: for the prime meridian and over the first set of frequencies and amplitudes, rough focusing the transducer on the target eye component, adjusting the position of the transducer on the target eye component, refocusing the transducer on the target eye component and repeating these operations as necessary to obtain a desired degree of image accuracy (step 1704); rotating the guide track to a secondary meridian (step 1708), and, for the secondary meridian and over the first set of frequencies and amplitudes, rough focusing the transducer on the target eye component, adjusting the position of the transducer on the target eye component, refocusing the transducer on the target eye component and repeating these operations as necessary to obtain a desired degree of image accuracy (step 1712). The processor then initiates a set of A-scans along each of the primary and secondary meridians (step 1716) and converts the A-scans for each of the primary and secondary meridian into a set of B-scans and processes the B-scans to form an image associated with each meridian (step 1720).

In step 1524, the processor 1528 next executes the ocular measurement instructions 1540 to determine one or more parameters of the target ocular component (e.g., one or more of the radii of curvature of the anterior and posterior cornea and lens; the cornea and lens thicknesses; angle between peripheral edges of the lens and cornea; an angle between a back of the sclera and/or cornea and the front of the iris, the trabecular-iris angle, and/or the on-axis distances between the anterior surface of the cornea to the anterior surface of the lens, and between the posterior surface of the lens to the anterior surface of the retina, the shape, dimension, or area of the ciliary body, the iris/pupil diameter ratio and/or a sclera feature. As will be appreciated, the radii of curvature of the cornea and lens, the iris/pupil ratio, sclera features, and shape or dimension of the ciliary body are a function of the IOP).

In step 1724, the processor 1528 next executes the measurement instructions 1540 to determine a selected parameter of a target or selected ocular component or structure and, in step 1728, identifies the ciliary body and/or trabecular mesh and optionally an initial IOP The ciliary body and/or trabecular mesh can be identified automatically and/or with user interface feedback from the clinician positioning a mouse cursor over the ciliary body or trabecular mesh in a displayed image of the patient's eye. In step 1732 of FIG. 17B, the processor 1528 executes the ocular treatment instructions 1544 and positions the transducer to ablate the ciliary body and/or vibrate the trabecular mesh.

In optional step 1736, the processor 1528 executes the ocular treatment instructions 1544 to cause the irradiating transducer to ablate the ciliary body over a different second range of ultrasound frequencies and amplitudes.

In optional step 1740, the processor 1528 executes the ocular treatment instructions 1544 to cause the irradiating transducer to emit ultrasound energy over a different third range of frequencies and amplitudes to vibrate the target ocular component (e.g., trabecular meshwork).

As will be appreciated, steps 1736 and 1740 can be executed alternatively or in combination with each other.

The processor 1528 again executes the imaging instructions 1536 and repeats steps 1704-1720.

In step 1748, the processor 1528 again executes the measurement instructions 1540 to determine a new (post-ablation) parameter of the target ocular component.

In step 1752, the processor 1528 executes the measurement instructions 1540 and compares the measurements from step 1724 to those of step 1748 and determines the changes or differences in the target ocular component parameter. As will be appreciated, the difference between the selected parameter before treatment and after treatment can be a function of the change in IOP before and after treatment.

In decision diamond 1756, the processor 1528 determines whether further treatment is required and, if so, one or more treatment parameters (e.g., which of steps 1736 and 1740 is to be performed), a frequency range of ablation or vibration, a duration of ultrasound energy emission during treatment, and the like). Depending on the decision, the processor 1528 either returns to step 1744 to cause further treatment or notifies the user (clinician) in step 1760 of the treatment results.

The first, second, and third frequency or wavelength or amplitude ranges are different. In some applications, the first, second, and third frequency or wavelength or amplitude ranges are nonoverlapping. In some applications, the mode, median, or mean frequency or wavelength or amplitude in each of the first, second, and third frequency ranges are different from one another. By way of illustration, each of the first, second, and third ranges has different mode, median, or mean frequencies; each of the first, second, and third ranges has different mode, median, or mean wavelengths; and each of the first, second, and third ranges has different mode, median, or mean amplitudes.

In one configuration, steps 1504 through 1524, comprise the following sub-operations: (a) the clinician fills the goggles 612, 707, or 1605 with saline fluid, (b) the scanning system acquires a first ultrasound image of a target ocular feature and measures radii of curvatures and distances (e.g., of the image before treatment or a normal image); (c) the processor or clinician sets Pₒᵤₜ about equal to Pᵢₙ by using a water column to increase Pₒᵤₜ; (d) the scanning system acquires a second ultrasound image of the target ocular feature (before treatment) and measures radii of curvatures and distances; (e) after ablation and trabecular meshwork vibration, Pᵢₙ is reduced and Pₒᵤₜ becomes greater than Pᵢₙ; (f) the scanning system acquires a third ultrasound image of the target ocular feature (after treatment) and measures radii of curvatures and distances; (g) the patient removes the goggles, Pout returns to less than Pin but the differential is less than prior natural state; and the processor sets the new difference to delta new where new (new IOP) is less than punt (initial IOP).

According to another embodiment, in an eye with elevated intraocular pressure, a method for reducing IOP uses ultrasound to 1) ablate the ciliary process which is the structure responsible for production of aqueous humor and to 2) vibrate or undulate the trabecular mesh to stimulate better drainage of fluid through the trabecular mesh and out of the eye can comprise the following steps:

where:

PIOP is the intraocular pressure

Poutside is the pressure outside the goggles

Pinside is the pressure inside the goggles but just outside the cornea of the eye

Pambient is the ambient pressure in the imaging/treatment room

Δp is the pressure difference PIOP−Pinside

In a first step, PIOP is measured with a goniometer (as an example, take PIOP as 788 mm Hg, which is high) So Δp=28 mm. Normal Δp is in the range 12 mm to 22 mm.

In a second step, with goggles (aka the ultrasound eye piece) on, fill goggles with saline fluid at Pambient. Thus Poutside=Pinside=Pambient=760 mm. So Δp=28 mm. Normal Δp is in the range 12 mm to 22 mm.

In a third step, take a first ultrasound image and measure the radii of curvatures and distances (this is an ultrasound image of the eye with elevated IOP).

In a fourth step, make Pinside higher so that it approaches PIOP by using a 6.25-inch high (159 mm) water column to increase Pinside by 12 mm Hg—this makes the pressure in the anterior segment of the eye now 772 mm. This should allow the eye to go back to its configuration with IOP in the normal range, as Δp=16 mm

In a fifth step, with goggles on, take a second ultrasound image and re-measure the radii of curvatures and distances (a normal ultra sound image with the whole eye at elevated IOP)

In a sixth step, with goggles on, ablate the ciliary process and vibrate or undulate the trabecular mesh. After ablation and vibration, release the pressure from the water column

In a seventh step, if PIOP is reduced to, say, 775 mm then Δp=15 mm which is within the normal range

In an eighth step, with goggles on, take a third ultrasound image and re-measure radii of curvatures and distances. These dimensions should be close to those of the second ultra sound image

In a ninth step, remove goggles, the new difference is Δpnew where Δpnew=15 mm and is less than Δpinitial which was 28 mm.

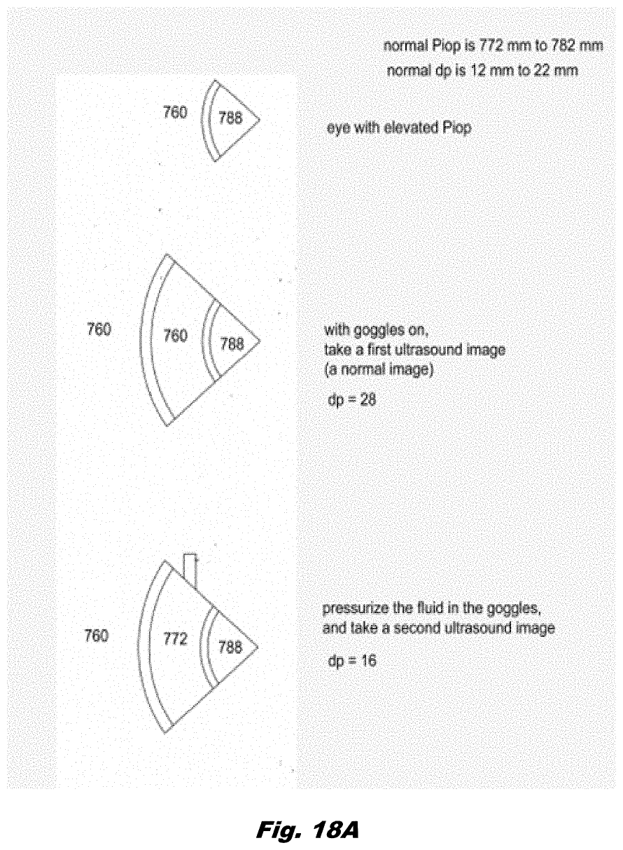

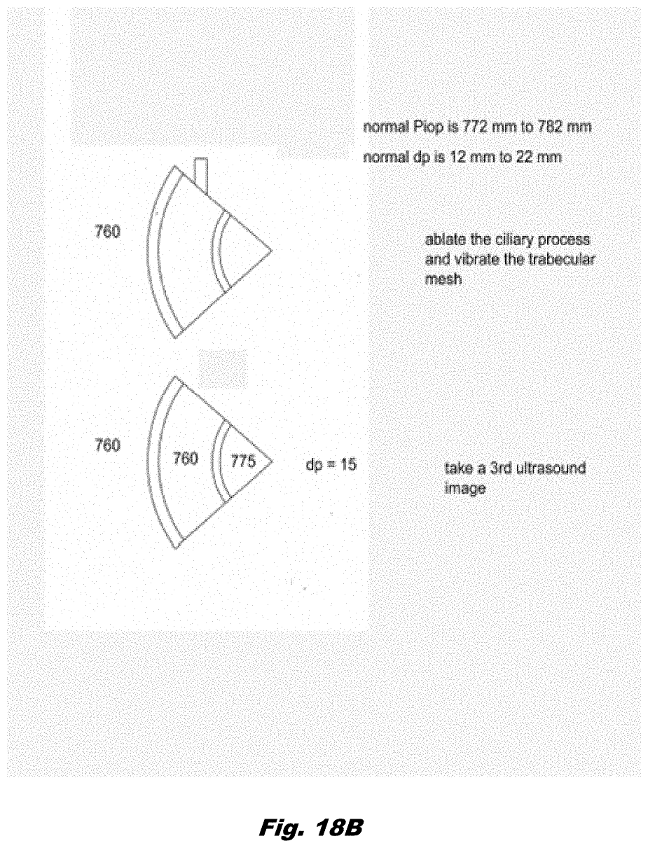

FIGS. 18A-B are a series of graphs summarizing pressures in various regions of the eye during the above nine steps of treatment and imaging.

This method can be implemented using a dual element or an annular array transducer or it can be implemented by separate single element transducers on a revolver type transducer mount.

In one embodiment, controller 1532 determines an adjustment to the position of the transducer and/or the OCT sample arm probe and the OCT reference arm based on receiving a control measurement input from the sensor array 1508. In another embodiment, controller 1532 provides a control input to the drive mechanism of the probe carriage, the positioning arm, the arcuate guide assembly, and/or the linear guide track. In yet another embodiment, controller 1532 provides a control input to comprise controlling the power, frequency, signal/noise ratio, pulse rate, gain schedule, saturation thresholds, and sensitivity of the optical and/or ultrasound transducers. In still another embodiment, controller 1532 utilizes control algorithms comprising at least one of on/off control, proportional control, differential control, integral control, state estimation, adaptive control and stochastic signal processing. Controller 1532 may also monitor and determine if any faults or diagnostic flags have been identified in one or more elements, such as the optical and/or ultrasound transducers and/or carriage.

In yet another embodiment, the disclosed systems and methods may be partially implemented in software that can be stored on a storage medium to include a computer-readable medium, executed on programmed general-purpose computer with the cooperation of a controller and memory, a special purpose computer, a microprocessor, or the like. In these instances, the systems and methods of this disclosure can be implemented as program embedded on personal computer such as an applet, JAVA® or CGI script, as a resource residing on a server or computer workstation, as a routine embedded in a dedicated measurement system, system component, or the like. The system can also be implemented by physically incorporating the system and/or method into a software and/or hardware system.

In one embodiment, one or more computers are used to control, among other things, the ultrasound imaging system, the scan head assembly, and/or the ultrasound transducer and/or the position sensor(s). In one embodiment, the user interacts with the computer through any means known to those skilled in the art, to include a keyboard and/or display to include a touch-screen display. The term “computer-readable medium” as used herein refers to any tangible storage and/or transmission medium that participate in providing instructions to a processor for execution. Such a medium may take many forms, including but not limited to, non-volatile media, volatile media, and transmission media. Non-volatile media includes, for example, NVRAM, or magnetic or optical disks. Volatile media includes dynamic memory, such as main memory. Common forms of computer-readable media include, for example, a floppy disk, a flexible disk, hard disk, magnetic tape, or any other magnetic medium, magneto-optical medium, a CD-ROM, any other optical medium, punch cards, paper tape, any other physical medium with patterns of holes, a RAM, a PROM, and EPROM, a FLASH-EPROM, a solid state medium like a memory card, any other memory chip or cartridge, a carrier wave as described hereinafter, or any other medium from which a computer can read. A digital file attachment to e-mail or other self-contained information archive or set of archives is considered a distribution medium equivalent to a tangible storage medium. When the computer-readable media is configured as a database, it is to be understood that the database may be any type of database, such as relational, hierarchical, object-oriented, and/or the like. Accordingly, the disclosure is considered to include a tangible storage medium or distribution medium and prior art-recognized equivalents and successor media, in which the software implementations of the present disclosure are stored.

A number of variations and modifications of the disclosed subject matter can be used. As will be appreciated, it would be possible to provide for some features of the disclosure without providing others.

The present disclosure, in various embodiments, includes components, methods, processes, systems and/or apparatus substantially as depicted and described herein, including various embodiments, sub-combinations, and subsets thereof. Those of skill in the art will understand how to make and use the present disclosure after understanding the present disclosure. The present disclosure, in various embodiments, includes providing devices and processes in the absence of items not depicted and/or described herein or in various embodiments hereof, including in the absence of such items as may have been used in previous devices or processes, for example for improving performance, achieving ease and\or reducing cost of implementation.

The foregoing discussion of the disclosure has been presented for purposes of illustration and description. The foregoing is not intended to limit the disclosure to the form or forms disclosed herein. In the foregoing Detailed Description for example, various features of the disclosure are grouped together in one or more embodiments for the purpose of streamlining the disclosure. This method of disclosure is not to be interpreted as reflecting an intention that the claimed disclosure requires more features than are expressly recited in each claim. Rather, as the following claims reflect, inventive aspects lie in less than all features of a single foregoing disclosed embodiment. Thus, the following claims are hereby incorporated into this Detailed Description, with each claim standing on its own as a separate preferred embodiment of the disclosure.

Moreover, though the description of the disclosure has included description of one or more embodiments and certain variations and modifications, other variations and modifications are within the scope of the disclosure, e.g., as may be within the skill and knowledge of those in the art, after understanding the present disclosure. It is intended to obtain rights which include alternative embodiments to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

What is claimed:

1. A system for imaging and treating an eye of a patient, comprising:
one or more ultrasound transducers;
a positioning mechanism that displaces the one or more ultrasound transducers into a desired location relative to an eye of the patient;
wherein:
in a first mode, the one or more ultrasound transducers emits ultrasound energy at a first range of frequencies to acquire an image of at least a portion of the eye of the patient; and
in a second mode, the one or more ultrasound transducers emits ultrasound energy at a second range of frequencies to alter a physical characteristic of the eye, wherein the first range of frequencies is different than the second range of frequencies.

2. The system of claim 1, wherein in the first and second modes the positioning mechanism is in a common spatial location, wherein the at least a portion of the eye comprises a ciliary body and/or trabecular meshwork, wherein the image of the at least a portion of the eye comprises an image of the ciliary body and trabecular meshwork, and wherein the physical characteristic of the eye is one or more of an intraocular pressure, radius of a cornea, radius of a lens, cornea thickness, lens thickness, angle between peripheral edges of the lens and cornea, angle between peripheral edges of the iris and cornea, on-axis distance between an anterior surface of the cornea and the anterior surface of the lens, on axis distance between a posterior surface of the cornea and the posterior surface of the lens, on axis distance between the posterior surface of the lens and the anterior surface of the retina, sclera parameter, and iris/pupil ratio.

3. The system of claim 2, wherein, in the first mode, the acquired image comprises a plurality of A-scan images of the at least a portion of an eye of a patient, and further comprising a processor that converts the plurality of A-scans to a plurality of B-scans, and wherein the plurality of B-scans comprise images of the ciliary body and trabecular meshwork, and wherein, in the second mode, the one or more transducers ablates at least a portion of the ciliary body and/or vibrates the trabecular mesh.

4. The system of claim 3, wherein a mode frequency of the first range of frequencies is different than a mode frequency of the second range of frequencies and further comprising:
a processor; and
a computer readable medium comprising a set of instructions that, when executed by the processor, cause the processor to:
determine, from a first image of the at least a portion of the eye, a first set of measurements, the first image being acquired before second mode and from a second image of the at least a portion of the eye, a second set of measurements, the second image being acquired after the second mode; and
compare the first and second sets of measurements to determine a degree of alteration of the physical characteristic of the eye.

5. The system of claim 1, wherein the physical characteristic of the eye comprises an angle between peripheral edges of the lens and cornea, an angle between a back of the sclera and/or cornea and the front of the iris, and/or a dimension or angle of the sclera, wherein in the first and second modes the one or more transducers have a commonly positioned focal point, wherein a median frequency of the first range of frequencies is different than a median frequency of the second range of frequencies and wherein a mean frequency of the first range of frequencies is different than a mean frequency of the second range of frequencies.

6. The system of claim 1, wherein the second mode comprises:
the one or more ultrasound transducers emitting ultrasound energy at the second range of frequencies to ablate a ciliary boy of the eye; and
the one or more ultrasound transducers vibrating a trabecular meshwork of the eye, wherein the second range of frequencies is different than the third range of frequencies.

7. A method for imaging and treating an eye of a patient comprising:
emitting, by one or more ultrasound transducers, ultrasound energy at a first range of wavelengths to acquire an image of at least a portion of an eye of a patient; and
thereafter, emitting, by the one or more ultrasound transducers, ultrasound energy at a second range of wavelengths to alter a physical characteristic of the eye, wherein the first range of wavelengths is different than the second range of wavelengths.

8. The method of claim 7, wherein the at least a portion of the eye comprises a ciliary body and/or trabecular meshwork, wherein the image of the at least a portion of the eye comprises an image of the ciliary body and trabecular meshwork, and wherein the physical characteristic of the eye is one or more of an intraocular pressure, radius of a cornea, radius of a lens, cornea thickness, lens thickness, angle between peripheral edges of the lens and cornea, an angle between a back of the sclera and/or cornea and the front of the iris, on-axis distance between an anterior surface of the cornea and the anterior surface of the lens, on axis distance between a posterior surface of the cornea and the posterior surface of the lens, on axis distance between the posterior surface of the lens and the anterior surface of the retina, sclera parameter, and iris/pupil ratio.

9. The method of claim 8, wherein the acquired image comprises a plurality of A-scan images of the at least a portion of an eye of a patient, and further comprising a processor that converts the plurality of A-scans to a plurality of B-scans, and wherein the plurality of B-scans comprise images of the ciliary body and trabecular meshwork, and wherein the one or more transducers ablates at least a portion of the ciliary body and/or vibrates the trabecular mesh.

10. The method of claim 9, wherein a mode frequency of the first range of frequencies is different than a mode frequency of the second range of frequencies and further comprising:
determining, from a first image of the at least a portion of the eye, a first set of measurements, the first image being acquired before second mode and from a second image of the at least a portion of the eye, a second set of measurements, the second image being acquired after the second mode; and
comparing the first and second sets of measurements to determine a degree of alteration of the physical characteristic of the eye.

11. The method of claim 8, wherein the physical characteristic of the eye comprises an angle between peripheral edges of the lens and cornea, an angle between a back of the sclera and/or cornea and the front of the iris, and/or a dimension or angle of the sclera, wherein in the first and second modes the one or more transducers have a commonly positioned focal point, wherein a median frequency of the first range of frequencies is different than a median frequency of the second range of frequencies and wherein a mean frequency of the first range of frequencies is different than a mean frequency of the second range of frequencies.

12. The method of claim 8, wherein the thereafter emitting comprises:
the one or more ultrasound transducers emitting ultrasound energy at the second range of frequencies to ablate a ciliary boy of the eye; and
the one or more ultrasound transducers vibrating a trabecular meshwork of the eye, wherein the second range of frequencies is different than the third range of frequencies.

13. A computer readable medium comprising instructions that, when executed by a processor, cause the processor to perform steps comprising:
emitting, by one or more ultrasound transducers, ultrasound energy at a first range of wavelengths to acquire an image of at least a portion of an eye of a patient; and
thereafter, emitting, by the one or more ultrasound transducers, ultrasound energy at a second range of wavelengths to alter a physical characteristic of the eye, wherein the first range of wavelengths is different than the second range of wavelengths.

14. The computer readable medium of claim 13, wherein the at least a portion of the eye comprises a ciliary body and/or trabecular meshwork, wherein the image of the at least a portion of the eye comprises an image of the ciliary body and trabecular meshwork, and wherein the physical characteristic of the eye is one or more of an intraocular pressure, radius of a cornea, radius of a lens, cornea thickness, lens thickness, angle between peripheral edges of the lens and cornea, on-axis distance between an anterior surface of the cornea and the anterior surface of the lens, on axis distance between a posterior surface of the cornea and the posterior surface of the lens, on axis distance between the posterior surface of the lens and the anterior surface of the retina, sclera parameter, and iris/pupil ratio.

15. The computer readable medium of claim 14, wherein the acquired image comprises a plurality of A-scan images of the at least a portion of an eye of a patient, and further comprising a processor that converts the plurality of A-scans to a plurality of B-scans, and wherein the plurality of B-scans comprise images of the ciliary body and trabecular meshwork, and wherein the one or more transducers ablates at least a portion of the ciliary body and/or vibrates the trabecular mesh.

16. The computer readable medium of claim 15, wherein a mode frequency of the first range of frequencies is different than a mode frequency of the second range of frequencies and further comprising the steps:
determining, from a first image of the at least a portion of the eye, a first set of measurements, the first image being acquired before second mode and from a second image of the at least a portion of the eye, a second set of measurements, the second image being acquired after the second mode; and
comparing the first and second sets of measurements to determine a degree of alteration of the physical characteristic of the eye.

17. The computer readable medium of claim 14, wherein the physical characteristic of the eye comprises an angle between peripheral edges of the lens and cornea, an angle between a back of the sclera and/or cornea and the front of the iris, and/or a dimension or angle of the sclera, wherein in the first and second modes the one or more transducers have a commonly positioned focal point, wherein a median frequency of the first range of frequencies is different than a median frequency of the second range of frequencies and wherein a mean frequency of the first range of frequencies is different than a mean frequency of the second range of frequencies.

18. The computer readable medium of claim 14, wherein the thereafter emitting comprises the substeps:
the one or more ultrasound transducers emitting ultrasound energy at the second range of frequencies to ablate a ciliary boy of the eye; and
the one or more ultrasound transducers vibrating a trabecular meshwork of the eye, wherein the second range of frequencies is different than the third range of frequencies.

19. An ultrasound device for an eye of a patient, comprising:
an eyepiece positioned near the eye, the eyepiece having an interior volume;
a window portion positioned on a surface of the eyepiece, the window portion being substantially parallel to the surface of the eye and substantially acoustically transparent, wherein the surface of the eyepiece is configured to operatively engage the eye of a patient;
a fluid disposed in the interior volume of the eyepiece;
a user interface to receive input from a user;
a processor;
a computer readable medium in communication with the processor; and
one or more ultrasound transducers positioned outside the fluid in the interior volume of the eyepiece, the one or more ultrasound transducers operably interconnected to at least one of an arcuate or linear track, wherein the instructions, when executed by the processor, cause the processor to operate in first and second modes:
in the first mode, the one or more ultrasound transducers emits ultrasound energy at a first range of frequencies to acquire an image of at least a portion of the eye of the patient, the at least a portion of the eye comprising the ciliary body and trabecular meshwork; and
in the second mode, the one or more ultrasound transducers emits ultrasound energy at a second range of frequencies to contact the ciliary body and/or trabecular meshwork to alter a physical characteristic of the eye, wherein the first range of frequencies is different than the second range of frequencies.
